SEQUENCE LISTING (104 sequences)

SEQ ID NO: 1 - protein, 4 aa, Artificial Sequence
  Description of Artificial Sequence Synthetic peptide
EAKA

SEQ ID NO: 2 - protein, 4 aa, Artificial Sequence
  Description of Artificial Sequence Synthetic peptide
RADA

SEQ ID NO: 3 - protein, 4 aa, Artificial Sequence
  Description of Artificial Sequence Synthetic peptide
DFAL

SEQ ID NO: 4 - protein, 6 aa, Artificial Sequence
  Description of Artificial Sequence Synthetic peptide
GAGAGS

SEQ ID NO: 5 - protein, 6 aa, Artificial Sequence
  Description of Artificial Sequence Synthetic peptide
GAGAGY

SEQ ID NO: 6 - protein, 12 aa, Artificial Sequence
  Description of Artificial Sequence Synthetic peptide
GDVGGAGATGGS

SEQ ID NO: 7 - protein, 30 aa, Artificial Sequence
  Description of Artificial Sequence Synthetic polypeptide
PPGPPGPPGPPGPPGPPGPPGPPGPPGPPG

SEQ ID NO: 8 - protein, 8 aa, Artificial Sequence
  Description of Artificial Sequence Synthetic peptide
NSGAITIG

SEQ ID NO: 9 - protein, 6 aa, Artificial Sequence
  Description of Artificial Sequence Synthetic peptide
GAITIG

SEQ ID NO: 10 - protein, 6 aa, Artificial Sequence
  Description of Artificial Sequence Synthetic peptide
VGVAPG

SEQ ID NO: 11 - protein, 4 aa, Artificial Sequence
  Description of Artificial Sequence Synthetic peptide
GQPR

SEQ ID NO: 12 - protein, 5 aa, Artificial Sequence
  Description of Artificial Sequence Synthetic peptide
KTTKS

SEQ ID NO: 13 - protein, 6 aa, Artificial Sequence
  Description of Artificial Sequence Synthetic peptide
VGVAPG

SEQ ID NO: 14 - protein, 7 aa, Artificial Sequence
  Description of Artificial Sequence Synthetic peptide
YYRADDA

SEQ ID NO: 15 - protein, 24 aa, Artificial Sequence
  Description of Artificial Sequence Synthetic peptide
EEEEEEEEEEEECCCCCCCCCCCC

SEQ ID NO: 16 - protein, 9 aa, Artificial Sequence
  Description of Artificial Sequence Synthetic peptide
ECECECECG

SEQ ID NO: 17 - protein, 5 aa, Artificial Sequence
  Description of Artificial Sequence Synthetic peptide
ECECG

SEQ ID NO: 18 - protein, 4 aa, Artificial Sequence
  Description of Artificial Sequence Synthetic peptide
CAAC

SEQ ID NO: 19 - protein, 4 aa, Artificial Sequence
  Description of Artificial Sequence Synthetic peptide
CACA

SEQ ID NO: 20 - protein, 4 aa, Artificial Sequence
  Description of Artificial Sequence Synthetic peptide
CCAA

SEQ ID NO: 21 - protein, 30 aa, Artificial Sequence
  Description of Artificial Sequence Synthetic polypeptide
GLKALEEKLKALEEKLKALEEKLKALEEKG

SEQ ID NO: 22 - protein, 12 aa, Artificial Sequence
  Description of Artificial Sequence Synthetic peptide
AGAGAGAGAGAG

SEQ ID NO: 23 - protein, 32 aa, Artificial Sequence
  Description of Artificial Sequence Synthetic polypeptide
AGAGAGAGAGAGAGAGAGAGAGAGAGAGAGAG

SEQ ID NO: 24 - protein, 8 aa, Artificial Sequence
  Description of Artificial Sequence Synthetic peptide
KLKLKLKL

SEQ ID NO: 25 - protein, 28 aa, Artificial Sequence
  Description of Artificial Sequence Synthetic peptide
KLKLKLKLKLKLKLKLKLKLKLKLKLKL

SEQ ID NO: 26 - protein, 18 aa, Artificial Sequence
  Description of Artificial Sequence Synthetic peptide
LKLLKKLLKKLKKLLKKL

SEQ ID NO: 27 - protein, 18 aa, Artificial Sequence
  Description of Artificial Sequence Synthetic peptide
KKKKKKKKKLLLLLLLLL

SEQ ID NO: 28 - protein, 18 aa, Artificial Sequence
  Description of Artificial Sequence Synthetic peptide
KLKLKLKLKLKLKLKLKL

SEQ ID NO: 29 - protein, 21 aa, Artificial Sequence
  Description of Artificial Sequence Synthetic peptide
KLAKKLAKLAKKLAKLAKKLA

SEQ ID NO: 30 - protein, 21 aa, Artificial Sequence
  Description of Artificial Sequence Synthetic peptide
KLAKLAKKLAKLAKKLAKLAK

SEQ ID NO: 31 - protein, 21 aa, Artificial Sequence
  Description of Artificial Sequence Synthetic peptide
KALKALKKALKALKKALKALK

SEQ ID NO: 32 - protein, 21 aa, Artificial Sequence
  Description of Artificial Sequence Synthetic peptide
KLGKKLGKLGKKLGKLGKKLG

SEQ ID NO: 33 - protein, 21 aa, Artificial Sequence
  Description of Artificial Sequence Synthetic peptide
KAAKKAAKAAKKAAKAAKKAA

SEQ ID NO: 34 - protein, 7 aa, Artificial Sequence
  Description of Artificial Sequence Synthetic peptide
SPRLQGV

SEQ ID NO: 35 - protein, 7 aa, Artificial Sequence
  Description of Artificial Sequence Synthetic peptide
TTLNPGT

SEQ ID NO: 36 - protein, 7 aa, Artificial Sequence
  Description of Artificial Sequence Synthetic peptide
VNSHPPL

SEQ ID NO: 37 - protein, 7 aa, Artificial Sequence
  Description of Artificial Sequence Synthetic peptide
TLPNHTF

SEQ ID NO: 38 - protein, 7 aa, Artificial Sequence
  Description of Artificial Sequence Synthetic peptide
QSVTSTK

SEQ ID NO: 39 - protein, 7 aa, Artificial Sequence
  Description of Artificial Sequence Synthetic peptide
PTSTGQA

SEQ ID NO: 40 - protein, 7 aa, Artificial Sequence
  Description of Artificial Sequence Synthetic peptide
TSPGQKQ

SEQ ID NO: 41 - protein, 7 aa, Artificial Sequence
  Description of Artificial Sequence Synthetic peptide
IGSSLKP

SEQ ID NO: 42 - protein, 14 aa, Artificial Sequence
  Description of Artificial Sequence Synthetic peptide
MHGKTQATSGTIQS

SEQ ID NO: 43 - protein, 14 aa, Artificial Sequence
  Description of Artificial Sequence Synthetic peptide
GLNDIFEAQKIEWH

SEQ ID NO: 44 - protein, 12 aa, Artificial Sequence
  Description of Artificial Sequence Synthetic peptide
AYSSGAPPMPPF

SEQ ID NO: 45 - protein, 12 aa, Artificial Sequence
  Description of Artificial Sequence Synthetic peptide
NPSSLFRTLPSD

SEQ ID NO: 46 - protein, 12 aa, Artificial Sequence
  Description of Artificial Sequence Synthetic peptide
SLATQPPRTPPV

SEQ ID NO: 47 - protein, 12 aa, Artificial Sequence
  Description of Artificial Sequence Synthetic peptide
MSPHPHPRHHHT

SEQ ID NO: 48 - protein, 12 aa, Artificial Sequence
  Description of Artificial Sequence Synthetic peptide
RGRRRRLSCRLL

SEQ ID NO: 49 - protein, 12 aa, Artificial Sequence
  Description of Artificial Sequence Synthetic peptide
KPSHHHHHTGAN

SEQ ID NO: 50 - protein, 20 aa, Artificial Sequence
  Description of Artificial Sequence Synthetic peptide
NTRMTARQHRSANHKSTQRA

SEQ ID NO: 51 - protein, 9 aa, Artificial Sequence
  Description of Artificial Sequence Synthetic peptide
YDSRSMRPH

SEQ ID NO: 52 - protein, 9 aa, Artificial Sequence
  Description of Artificial Sequence Synthetic peptide
VVRPKAATN

SEQ ID NO: 53 - protein, 9 aa, Artificial Sequence
  Description of Artificial Sequence Synthetic peptide
RIRHRLVGQ

SEQ ID NO: 54 - protein, 9 aa, Artificial Sequence
  Description of Artificial Sequence Synthetic peptide
GRMQRRVAH

SEQ ID NO: 55 - protein, 9 aa, Artificial Sequence
  Description of Artificial Sequence Synthetic peptide
LGKDRPHFH

SEQ ID NO: 56 - protein, 7 aa, Artificial Sequence
  Description of Artificial Sequence Synthetic peptide
AAAAAAD

SEQ ID NO: 57 - protein, 7 aa, Artificial Sequence
  Description of Artificial Sequence Synthetic peptide
VVVVVVD

SEQ ID NO: 58 - protein, 7 aa, Artificial Sequence
  Description of Artificial Sequence Synthetic peptide
KAAAAAA

SEQ ID NO: 59 - protein, 7 aa, Artificial Sequence
  Description of Artificial Sequence Synthetic peptide
GAVILRR

SEQ ID NO: 60 - protein, 4 aa, Artificial Sequence
  Description of Artificial Sequence Synthetic peptide
IIIK

SEQ ID NO: 61 - protein, 4 aa, Artificial Sequence
  Description of Artificial Sequence Synthetic peptide
LLLK

SEQ ID NO: 62 - protein, 17 aa, Artificial Sequence
  Description of Artificial Sequence Synthetic peptide
XXXXXHHHHRRRRRRRR

SEQ ID NO: 63 - protein, 20 aa, Artificial Sequence
  Description of Artificial Sequence Synthetic peptide
HHHHHHHHHHRRRRRRRRRR

SEQ ID NO: 64 - protein, 32 aa, Artificial Sequence
  Description of Artificial Sequence Synthetic polypeptide
AFAFAFAFAFAFHHHHHKKKKKKKKKKKKKKK

SEQ ID NO: 65 - protein, 32 aa, Artificial Sequence
  Description of Artificial Sequence Synthetic polypeptide
AAAAAAAAAAAAHHHHHKKKKKKKKKKKKKKK

SEQ ID NO: 66 - protein, 15 aa, Artificial Sequence
  Description of Artificial Sequence Synthetic peptide
AAVVLLLWEEEEEEE

SEQ ID NO: 67 - protein, 7 aa, Artificial Sequence
  Description of Artificial Sequence Synthetic peptide
EVHHQKL

SEQ ID NO: 68 - protein, 11 aa, Artificial Sequence
  Description of Artificial Sequence Synthetic peptide
CCCCGGGSRGD

SEQ ID NO: 69 - protein, 11 aa, Artificial Sequence
  Description of Artificial Sequence Synthetic peptide
AAAAGGGSRGD

SEQ ID NO: 70 - protein, 13 aa, Artificial Sequence
  Description of Artificial Sequence Synthetic peptide
AAAAGGGEIKVAV

SEQ ID NO: 71 - protein, 8 aa, Artificial Sequence
  Description of Artificial Sequence Synthetic peptide
AAAAKKKK

SEQ ID NO: 72 - protein, 11 aa, Artificial Sequence
  Description of Artificial Sequence Synthetic peptide
CCCCGGGSRGD

SEQ ID NO: 73 - protein, 17 aa, Artificial Sequence
  Description of Artificial Sequence Synthetic peptide
WAAAAKAAAAKAAAAKA

SEQ ID NO: 74 - protein, 5 aa, Artificial Sequence
  Description of Artificial Sequence Synthetic peptide
XVEVE

SEQ ID NO: 75 - protein, 9 aa, Artificial Sequence
  Description of Artificial Sequence Synthetic peptide
VVVAAAEEE

SEQ ID NO: 76 - protein, 13 aa, Artificial Sequence
  Description of Artificial Sequence Synthetic peptide
VVVAAAKKKRGDS

SEQ ID NO: 77 - protein, 16 aa, Artificial Sequence
  Description of Artificial Sequence Synthetic peptide
LSQETFSDLWKLLPEN

SEQ ID NO: 78 - protein, 13 aa, Artificial Sequence
  Description of Artificial Sequence Synthetic peptide
GTAGLIGQERGDS

SEQ ID NO: 79 - protein, 30 aa, Artificial Sequence
  Description of Artificial Sequence Synthetic polypeptide
GGGRRRRRRYGRKKRRQRRRRGDSKKLLAK

SEQ ID NO: 80 - protein, 5 aa, Artificial Sequence
  Description of Artificial Sequence Synthetic peptide
KTTKS

SEQ ID NO: 81 - protein, 4 aa, Artificial Sequence
  Description of Artificial Sequence Synthetic peptide
GQPR

SEQ ID NO: 82 - protein, 25 aa, Artificial Sequence
  Description of Artificial Sequence Synthetic peptide
AXAEAAEKAAKYAAEAAEKAAKAXA

SEQ ID NO: 83 - protein, 12 aa, Artificial Sequence
  Description of Artificial Sequence Synthetic peptide
AGAGAGAGAGAG

SEQ ID NO: 84 - protein, 14 aa, Artificial Sequence
  Description of Artificial Sequence Synthetic peptide
AGAGAGAGAGAGAG

SEQ ID NO: 85 - protein, 16 aa, Artificial Sequence
  Description of Artificial Sequence Synthetic peptide
AGAGAGAGAGAGAGAG

SEQ ID NO: 86 - protein, 18 aa, Artificial Sequence
  Description of Artificial Sequence Synthetic peptide
AGAGAGAGAGAGAGAGAG

SEQ ID NO: 87 - protein, 20 aa, Artificial Sequence
  Description of Artificial Sequence Synthetic peptide
AGAGAGAGAGAGAGAGAGAG

SEQ ID NO: 88 - protein, 22 aa, Artificial Sequence
  Description of Artificial Sequence Synthetic peptide
AGAGAGAGAGAGAGAGAGAGAG

SEQ ID NO: 89 - protein, 24 aa, Artificial Sequence
  Description of Artificial Sequence Synthetic peptide
AGAGAGAGAGAGAGAGAGAGAGAG

SEQ ID NO: 90 - protein, 26 aa, Artificial Sequence
  Description of Artificial Sequence Synthetic peptide
AGAGAGAGAGAGAGAGAGAGAGAGAG

SEQ ID NO: 91 - protein, 28 aa, Artificial Sequence
  Description of Artificial Sequence Synthetic peptide
AGAGAGAGAGAGAGAGAGAGAGAGAGAG

SEQ ID NO: 92 - protein, 30 aa, Artificial Sequence
  Description of Artificial Sequence Synthetic polypeptide
AGAGAGAGAGAGAGAGAGAGAGAGAGAGAG

SEQ ID NO: 93 - protein, 32 aa, Artificial Sequence; identical to SEQ ID NO: 23
  Description of Artificial Sequence Synthetic polypeptide

SEQ ID NO: 94 - protein, 8 aa, Artificial Sequence
  Description of Artificial Sequence Synthetic peptide
KLKLKLKL

SEQ ID NO: 95 - protein, 10 aa, Artificial Sequence
  Description of Artificial Sequence Synthetic peptide
KLKLKLKLKL

SEQ ID NO: 96 - protein, 12 aa, Artificial Sequence
  Description of Artificial Sequence Synthetic peptide
KLKLKLKLKLKL

SEQ ID NO: 97 - protein, 14 aa, Artificial Sequence
  Description of Artificial Sequence Synthetic peptide
KLKLKLKLKLKLKL

SEQ ID NO: 98 - protein, 16 aa, Artificial Sequence
  Description of Artificial Sequence Synthetic peptide
KLKLKLKLKLKLKLKL

SEQ ID NO: 99 - protein, 18 aa, Artificial Sequence
  Description of Artificial Sequence Synthetic peptide
KLKLKLKLKLKLKLKLKL

SEQ ID NO: 100 - protein, 20 aa, Artificial Sequence
  Description of Artificial Sequence Synthetic peptide
KLKLKLKLKLKLKLKLKLKL

SEQ ID NO: 101 - protein, 22 aa, Artificial Sequence
  Description of Artificial Sequence Synthetic peptide
KLKLKLKLKLKLKLKLKLKLKL

SEQ ID NO: 102 - protein, 24 aa, Artificial Sequence
  Description of Artificial Sequence Synthetic peptide
KLKLKLKLKLKLKLKLKLKLKLKL

SEQ ID NO: 103 - protein, 26 aa, Artificial Sequence
  Description of Artificial Sequence Synthetic peptide
KLKLKLKLKLKLKLKLKLKLKLKLKL

SEQ ID NO: 104 - protein, 28 aa, Artificial Sequence
  Description of Artificial Sequence Synthetic peptide
KLKLKLKLKLKLKLKLKLKLKLKLKLKL

(19) United States
(12) Patent Application Publication
(10) Pub. No.: US 2012/0282652 A1
(43) Pub. Date: Nov. 8, 2012

(54) PREPARATION OF PEPTIDE MIXTURES BY PROTEASE CATALYSIS DESIGNED TO PROVIDE USEFUL BIOLOGICAL AND PHYSICAL PROPERTIES

(75) Inventors: Richard A. Gross, Plainview, NY (US); Kodandaraman Viswanathan, Chennai (IN); Xu Qin, Brooklyn, NY (US)

(73) Assignee: Polytechnic Institute of New York University, Brooklyn, NY (US)

(21) Appl. No.: 13/406,751

(22) Filed: Feb. 28, 2012

Related U.S. Application Data
(60) Provisional application No. 61/447,322, filed on Feb. 28, 2011.

(51) Int. Cl. C12P 21/02

(52) U.S. Cl. 435/68.1

(57) ABSTRACT

A process for preparing unique peptide mixtures with a broad range of uses using combinations of natural and non-natural amino acid alkyl ester monomers and specific combinations of these monomers as dimers, trimers and higher oligomers selected from the large group of structural motifs that lead to useful physical and/or biological properties and that have been or may be identified from solid state peptide synthesis, isolation of peptides from natural sources, and production of peptides by recombinant DNA methods, or identification of peptides by recombinant methods such as phage display, the method of peptide synthesis comprising a) admixing one or more natural and non-natural amino acid alkyl ester monomer, dimer, trimer and higher oligomers with one or more proteases in a reaction medium; b) heating the mixture to between about 5° C. to about 90° C. for between 5 minutes and 24 hours; and c) recovering the formed oligopeptide.

### STATEMENT OF RELATED APPLICATIONS

This patent application claims priority on and the benefit of U.S. Provisional Patent Application No. 61/447,322 having a filing date of 28 Feb. 2011.

### BACKGROUND OF THE INVENTION

1. Technical Field

This invention relates to the synthesis and use of unique peptide mixtures. Protease-catalyzed oligomerization is used to prepare peptide mixtures from natural and non-natural amino acid ester monomers and specific combinations of these monomers as dimers, trimers and higher oligomers. Incorporated in peptide mixtures are structural motifs that mimic those found in nature, or structural motifs discovered by other peptide synthetic methods.

2. Prior Art

Peptide synthesis and the development of applications for oligopeptides are concomitantly evolving fields of science and technology. One example is the use of oligopeptides for metal binding applications. Oligopeptides with specific amino acid sequences have been used to bind heavy metals such as Cd(II), Co(II), Zn(II), Ni(II), Pb(II), Cu(II), etc. An important function of metal binding oligopeptides is to reduce or eliminate toxic effects on biological systems when heavy metal concentrates exceed safe levels.¹ In nature, there are a variety of responses to heavy metal contamination. For example, gram positive bacteria have evolved a system of uptake and detoxification in which a cysteine-containing mercury binding peptide (merP) is expressed.² In plants, heavy metals are eliminated by a unique metal sequestration mechanism involving phytochelatin (PC) peptides as a product of their biosynthetic pathway, under the action of an enzyme known as PC synthase.³ PC's are one example of structural motifs found in nature that are incorporated in this invention by mimicking their structure by protease-catalyzed oligomerization of natural and non-natural amino acid ester monomers and specific combinations of these monomers as dimers, trimers and higher oligomers.

The high affinity of PC binding to soft divalent metals is attributed to the structural motif of cysteine thiolate groups spaced at even intervals.³ The primary structures of phytochelatin and their variants have been identified in the review by W E Rauser.⁴ The presence of structurally organized domains, which usually contain equally spaced cysteine units responsible for binding Zn(II), has also been documented in the review by Berg.⁵ Application areas include developing novel phytoremediation methods wherein naturally occurring metal hyper-accumulators are used to remove toxic metals from contaminated soils.⁶

Many authors have investigated the chemical structures of PCs responsible for their metal binding property.³ One common motif is the γGlu-Cys or (γGlu-Cys)ₙ-Xaa (Xaa=variable amino acid such as Ser, Glu, Gly, Ala), in which the glutamic acid unit is γ-linked to the cysteine by an amide bond. All these studies used solid phase peptide synthesis as shown in Chart 1.^{3, 7, 8} Table 1 describes representative examples of sequences that result in peptides that bind inorganic compounds.

Conventional antibiotics usually overcome bacteria by targeting specific molecules that are part of the bacterial cellular machinery. Widespread use of these agents has caused rapid development of antibacterial drug resistance which is a critical issue in the treatment of infectious diseases. The evolution of antibiotic-resistant bacteria has stimulated the search for new potential bactericidal agents for which antimicrobial peptides are considered as promising candidates with broad-spectrum activity.⁹ Industrial development of these peptides is hampered by high costs associated with multiple protection and de-protection steps involved in their large scale production by conventional chemical methods. Isolation of these components from natural sources is also tedious.

α-Helical antimicrobial peptides (AMPs) have primarily been isolated from natural sources and their sequence signatures can be obtained by using a modified version of Prosite (http://www.expasy.ch/prosite). Furthermore, the sequence information of several 100 different peptides has been characterized and a comprehensive list of reviews and publications are available (see below).

The two common and functionally important requirements of AMPs are as follows: i) a net cationic charge for interaction with negatively charged microbial surfaces, ii) ability to assume amphipathic structures that permit incorporation into microbial membranes.¹⁰

The mechanism by which antibiotic peptides function is still under considerable debate. However, all evidence indicates that antimicrobial peptides act by permeabilizing microbial cell membranes^{11, 12} which results in leakage of cellular contents and, eventually, cell death. Many AMPs share three structural traits that facilitate their ability to interact with and perturb anionic pathogenic membranes: 1) the presence of positively charged residues, 2) a large proportion of hydrophobic residues¹³, and 3) an ability to adopt a shape in which clusters of hydrophobic and cationic amino acids are spatially organized in discrete sectors of the molecule (‘amphipathic’ design)¹⁴.

Most often, the preparation of peptides with an alternating sequence of amino acids is performed by solid phase or liquid phase peptide synthesis methods.¹⁵ However, these chemical synthetic methods involve multiple steps with extensive protection-deprotection chemistry. Since it is rather difficult to recycle the large excess of the coupling reagent and acyl donor involved in synthesis, the overall efficiency can be rather low. Also, the toxic nature of the solvents and the coupling reagents employed leads to obvious health and environmental concerns.¹⁶

Protein engineering of alternating peptide sequences into organisms and subsequent production of peptides via fermentation provides an alternative route to prepare precise sequence and chain length products. While this approach is promising, peptide yields via fermentation are generally low and purification requires specialized methods.¹⁷ Synthesis of alternating sequences using genetic engineering methods often remains impractical due to the low expression efficiencies obtained and difficulties encountered in product extraction and recovery.¹⁸

Another commonly practiced route to synthesize poly(α-amino acid)s of high molecular weight is by ring-opening polymerization of α-amino acid N-carboxylic anhydrides (NCAs). While NCA polymerization can be used for random or block copolymers, it is not useful for attaining specific sequences of two or more amino acids such as are present in alternating co-oligopeptides.¹⁹ Moreover, NCA ring-opening polymerization uses toxic phosgene or their equivalents for monomer synthesis, and requires strict removal of water and high monomer purity.

Cationic antibacterial peptides are defined as peptides containing a net excess of positively charged residues and approximately 50% hydrophobic residues. Based on the concept that cationic charge and lipophilicity are major factors determining antibacterial activity in oligopeptides, synthetic antimicrobial peptides (AMPs) with alternating Arg-Trp sequence containing one to five repeats were tested for bactericidal activity in plasma. This family of RW-series alternating peptides shows high activity on most of bacteria tested.^{20, 21}

Peptides with amino acid sequences of alternating hydrophobic and hydrophilic side groups could self-assembly to form stimulus-responsive hydrogel. This new class of peptide-based biomaterials has been actively pursued as a molecular-engineered scaffold for tissue repair.²² Zhang and co-workers have developed a series of peptides that form stable hydrogels at low peptide concentrations (0.1-1%). They are characterized by an alternating sequence of hydrophobic and hydrophilic residues, in which the hydrophilic residues, in turn, alternate between being positively and negatively charged, such as in (KLD)ₙ, (EAKA)ₙ and (RADA)ₙ. The alternation between polar and non-polar residues promotes the formation of a β-strand building block with hydrophobic and hydrophilic faces. These types of peptides have been shown to be non-cytotoxic and of potential use in the repair of cartilage tissue.²³

Liquid Phase Peptide Synthesis (LPPS) has been used to synthesize small peptides, such as dipeptides and their derivatives. By using different coupling agents, peptide bonds are synthesized from the N-terminal amine of amino acid A and C-terminal carboxylic acid of amino acid B while all other groups on both amino acids are protected using selected groups. This technique is highly flexible with respect to the chemistry of coupling and the combination of the peptidic blocks. New strategies for synthesis in solution have been developed, going from the design of functional groups for the side chains to the use of new coupling reagents.²⁴

While Liquid Phase Peptide Synthesis (LPPS) dominates synthesis of short (e.g. 2 or 3 amino acids in length) peptides of uniform sequence and chain length, enzyme-catalysis is also used. Recent examples are the synthesis of a series of histidine containing dipeptides catalyzed by trypsin and chymotrypsin; the formation of several dipeptides by a new thermophilic protease from Clostridium thermohydrosulfuricum; the pepsin-catalyzed synthesis of various model peptides; the use of Pronase from S. griseus as a catalyst for dipeptide synthesis; the preparation of short peptides such as, e.g., kyotorphin (H-Tyr-Arg-OH) starting from H-Tyr-OEt and Arg-OH or H-Asp-Phe-Ala-Leu-OH by condensation of H-Asp-Phe-OMe and H-Ala-Leu-OH, and various peptide esters in frozen aqueous media.²⁵

Preliminary results demonstrated that, in many cases, co-oligomerization of two different amino acid ethyl ester monomers results in random oligopeptides. For this reason, an alternative route was developed that involves a first step in which dipeptides are first synthesized chemically followed by an enzyme-catalyzed oligomerization of dipeptides to prepare alternating oligopeptides. Using this approach, the alternating peptides (Ala-Gly)ₓ, (Lys-Leu)ₓ and (Lys-Phe)ₓ were prepared.

Most antimicrobial peptides exhibit a net positive charge at pH values up to 9.²⁶ This cationic nature is largely due to a significant number of arginine and lysine residues within the amino acid sequence of peptides. The resulting net positive charge enhances electrostatic interactions between AMPs and the anionic bacterial membranes. Moreover, this positive charge helps enhance selectivity for pathogenic membranes over host cell membranes because the latter are most often net neutral.

The hydrophobicity of AMPs is defined as the percentage of hydrophobic residues within the peptide's primary structure. Although hydrophobicity is necessary for effective membrane permeabilization, AMPs with increased levels of hydrophobic character exhibit an attenuated selectivity for pathogens as well as an increased level of toxicity towards mammalian cells¹³.

Many AMPs with extended structures (e.g. helical) contain a high population of specific amino acids such Pro, Trp, Arg and His²⁷. Such structural motifs in AMPs minimize structural constraints and enhance the tendency of AMPs to adopt more extended structures. Examples include the Trp- and Pro-rich peptide indolicidin and the His-rich peptide histatin.

It is well known to those skilled in the art based on literature reports that specific sequences or structural motifs show binding to different noble metals and noble metal oxides (see Table 1).²⁸ The presence of a Cys₂-His₂ sequence is most prevalent in metal binding proteins (especially in zinc finger proteins).²⁹ Similarly there are certain identifiable sequences that mimic silk properties. Illustrations of such sequences include one identified for the peptide fibroin C (GAGAGS) as well as silk sequences for fibroin A (GAGAGY) and fibroin V (GDVGGAGATGGS) that lead to amorphous silk domains.³⁰

Peptides can be constructed that mimic collagen properties that have the repeat sequence (Pro-Hyp-Gly)₁₀ where Hyp corresponds to hydroxyproline.³¹ Peptide can also be constructed that self assemble to higher order structures by building peptides from known peptide sequences with this property. For example, amyloid peptides have structural motifs or sequences of the type NSGAITIG and GAITIG.³² Both sequences not only show amyloidogenic properties but can also bind gold, silver and platinum nanoparticles.³² Another property of importance is cell adhesion; many adhesive peptides possess RGD repeat sequences.³³

Furthermore, some examples of peptide motifs found in peptides useful in cosmetic properties are as follows: palmitoyl hexapeptide, Pal-VGVAPG-OH, palmitoyl tetrapeptide Pal-GQPR, and palmitoyl pentapeptide which is a derivative of pro-collagen I pentapeptide KTTKS (source: http://www.articlesbase.com/medicine-articles/cosmetic-peptides-2201766.html).

Peptides are of great interest as cosmeceuticals for applications such as treating aging skin. Methods described herein for synthesis of peptide mixtures can be used to prepare products that provide the benefits of peptide cosmeceuticals but at much lower costs relative to conventional peptide synthesis methods currently used. There are three main categories of cosmeceutical peptides: signal peptides, neurotransmitter-affecting peptides and carrier peptides. A valine-glycine-valine-alanine-proline-glycine (VGVAPG) peptide was discovered to significantly stimulate human dermal skin fibroblast production, while another study examined inhibition of procollagen-C proteinase by the peptide tyrosine-tyrosine-arginine-alanine-aspartame-aspartame-alanine (YYRADDA) leading to decreased collagen breakdown.³⁴ Another important example of a peptide used in cosmeceutical applications is the signal peptide sequence lysine-threonine-threonine-lysine-serine (KTTKS) found on type I procollagen. This pentapeptide stimulates feedback regulation for new collagen synthesis resulting in increased production of extracellular matrix proteins such as types I and II collagen and fibronectin.³⁵

Short synthetic peptide amphiphiles have recently been explored as effective nanobiomaterials in applications ranging from controlled gene and drug release, skin care, nanofabrication, biomineralization, membrane protein stabilization to 3D cell culture and tissue engineering. Many self-assembled materials consisting of peptide amphiphiles show sensitive responses to changes in environmental conditions such as pH, temperature, ionic strength, different ions, and light.³⁶ Self-assembling peptides have been designed to form different structured aggregates such as nanofibers, nanovesicles, nanobelts, and nanotubes. They possess a hydrophobic tail and a hydrophilic head. The tails of peptide amphiphiles are normally composed of non-polar amino acid residues (G, A, V, I, L, P and F). These amino acids have different size, shape and hydrophobicity. Meanwhile, the tails can also be made of hydrocarbon chains or even a mixture of a hydrocarbon chain and non-polar amino acids. The hydrophilic heads of these molecules can also be positively charged (H, K and R), negatively charged (D and E) or contain combinations of both. Representative amphiphilic peptides are listed in the Table 2.³⁷

### BRIEF SUMMARY OF THE INVENTION

It is the objective of the present invention to mitigate the aforementioned disadvantages and to provide simpler methods to large scale production of peptides with useful biological activities or physical properties such as metal binding.

To avoid chemically intensive syntheses involving multiple steps of protection and de-protection performed when peptides are prepared by solid or liquid phase synthesis, this invention describes a unique approach that avoids chemically intense processes to prepare oligopeptides with metal-binding activity. This was accomplished by mimicking a structural motif found in PCs so that they occur within peptide mixtures produced by protease-catalyzed oligomerization of amino acid alkyl ester monomers, dimers, trimers or higher oligomers. Table 1 describes representative examples of sequences that result in peptides that bind inorganic compounds.

The methods described in this invention can be used to prepare peptide amphiphiles that have the valued properties of those described above. However, by replacing conventional peptide synthetic methods with protease-catalyzed oligopeptide synthetic methods described herein, the cost of the above products will be greatly reduced. One example illustrated herein uses protease catalysis in combination with chemical modification to produce amphiphilic lipopeptides.

This invention discloses a unique approach that avoids chemically intense processes in order to prepare AMPs. This was accomplished by mimicking AMP structural motifs so that they occur within peptide mixtures produced by protease-catalyzed oligomerization of amino acid ester monomers and specific combinations of these monomers as dimers, trimers and higher oligomers. As an example of this invention, structural motifs that many AMP's share that include the presence of positively charged residues and a large proportion of hydrophobic residues was obtained in peptide mixtures by protease-catalyzed co-oligomerization of Lys and Leu alkyl ester mixtures. In this way, a family of lys-co-leu amphiphilic AMP's with Lys/Leu ratios from 1/1 to 2/1, and chain lengths as long as 20 residues were prepared. Antimicrobial assays performed on AMP's prepared by protease-catalyzed oligomerization of Lys and Leu alkyl ester mixtures verified that, by this simple and cost-effective strategy, a unique mixture of oligopeptides was produced that displayed useful antimicrobial activity.

Another example illustrating this invention is Arg-co-Trp peptides. Synthesis of Arg-co-Trp peptides by solid phase synthesis with specific sequences and chain lengths (from 6 to 10) has demonstrated structural motifs that give extended structures with substantial antimicrobial activities. Industrial development of Arg-co-Trp peptides has been hampered by high costs associated with multiple protection and de-protection steps involved in their production by conventional chemical methods. In this invention we disclose that Arg-co-Trp peptides can be prepared so that the structural motifs that result in their antimicrobial activity occur within peptide mixtures produced by protease-catalyzed oligomerization of amino acid alkyl ester monomers and specific combinations of these monomers as dimers, trimers and higher oligomers. Thus, a family of Arg-co-Trp AMP's with Arg/Trp ratios from 1/1 to 1/2 were prepared. Antimicrobial assays performed on Arg-co-Trp AMP's prepared by protease-catalyzed oligomerization verified that, by this simple and cost-effective strategy, a unique mixture of oligopeptides were prepared with useful antimicrobial activity.

Silks are a widely studied family of naturally occurring structural proteins. They are considered to be nature's high-performance material due to their remarkable combination of strength and toughness. These mechanical properties stem from the protein secondary structure that consists of a combination of well defined β-sheets in a less well-defined glycine-rich matrix. To translate this structure into a synthetic material, peptides containing the structural motif of alternating alanine-glycine units have been investigated. The observation that alanylglycine (AG) repeating units form extended β strands in a variety of AG rich polypeptides, including Bombyx mori silk fibroin,³⁸ was the basis for the preparation of polypeptides containing this repetitive sequence. By using atom-transfer radical polymerization (ATRP) and a bifunctional initiator, a well-defined β-sheet side chain peptide-based polymer was prepared.³⁹ The preparation and assembly of a triblock copolymer consisting of a central β-sheet polypeptide block composed of the repetitive (AG)ₙ sequence conjugated to PEG end blocks was also reported.⁴⁰ This invention discloses a unique approach by which AG repeat unit oligopeptides are prepared by protease-catalyzed oligomerization of AG dimer into longer chain alternating dipeptides. This method is much simpler and lower cost than currently used chemical synthetic peptide methods such as solid-state synthesis.

Inherent advantages of enzymatic peptide synthesis has led to its evolution as an alternative to chemical coupling methods.⁴¹ The thiol-protease papain is reported to be the most efficient catalyst for aqueous phase synthesis of homo-oligomers of hydrophobic amino acids like leucine, methionine, phenylalanine, and tyrosine.^{42, 43} The equilibrium of such reactions is tilted in favor of synthesis by the precipitation of hydrophobic oligomers. In examples given in this invention, protease concentrations were normalized based on a common activity unit.⁴⁴⁻⁴⁶ The casein assay was adopted to define the hydrolytic activity of different proteases used in this invention.⁴⁷ An alternative assay method to quantify protease activity can be substituted for the casein assay and such methods are well known by persons of ordinary skill in the art. This overcomes the problem of using protease catalysts whose activity is not quantified in a way that would allow others skilled in the art to repeat their work.

There are a wide range of peptide sequences or structural motifs that are known to one skilled in the art that provide various types of bioactivity (e.g. antibacterial, antifungal, protein inhibition and antihypertensive, antioxidant, calcium binding, prevent aggregation of platelets, chelate metals or provide other valuable properties. This invention discloses a method by which the beneficial biological and/or physical properties of these peptides are obtained to various extents by incorporating structural motifs of said peptides within peptide mixtures by producing said peptides by protease-catalyzed oligomerization of amino acid alkyl ester monomers and specific combinations of these monomers as dimers, trimers and higher oligomers.

Heavy metals are eliminated from plants by a unique metal sequestration mechanism involving phytochelatin (PC) peptides. PCs are one example of peptides with structural motifs that can be replicated by the methods disclosed in this invention. That is, the metal binding properties of PC's can be replicated to various extents by the judicious selection of amino acid alkyl ester monomers and specific combinations of these monomers as dimers, trimers and higher oligomers that are oligomerized by protease-catalysis. This was demonstrated by conducting the papain-catalyzed co-oligomerization of glutamic acid diethyl ester hydrochloride [γ-L-(Et)-2-Glu.HCl] and cysteine ethyl ester hydrochloride [L-Et-Cys.HCl]. By varying the feed ratio of the two co-monomers, synthesis of a ˜1:1 α-linked oligo(γ-L-Et-Glu-co-L-Cys) was achieved under buffered aqueous conditions. Following hydrolysis of the side chain ethyl ester groups of α-linked L-glutamic acid, it was demonstrated by a series of methods that included UV-VIS spectroscopy, ¹H-NMR and CD spectropolarimetry that the resulting water-soluble oligo(L-Glu-co-L-Cys) peptides that mimic PC's have useful binding affinities to several divalent heavy metals [Zn(II), Cd(II), Co(II), Ni(II)].

Previous publications demonstrate that other commercially available proteases that include but are not limited to bromelain, α-chymotrypsin and trypsin, can efficiently synthesize peptide bonds by oligomerization of natural amino acid ethyl ester monomers⁴⁸. As an example of this invention, the reaction pH and feeding ratio of L-Lys ethyl ester and L-Leu ethyl ester was varied to produce in both aqueous buffer and pure water media oligo(L-lys-co-L-Leu) with Lys/Leu ratios from 1:1 to 1:4 and chain lengths up to 15. The resulting precipitate was washed to remove proteases and the antimicrobial activity of peptides was confirmed from assays conducted with various pathogens based on inhibition of microbe growth.

AMP's consisting of L-lys-co-L-Leu and L-Arg-co-L-Leu, produced by protease-catalysis from amino acid alkyl ester monomers, dimers, trimers or higher oligomers can be fractionated to obtain products with correspondingly higher antimicrobial activity on target microorganisms. Various fractions of products were characterized to determine oligopeptide composition by ¹H-NMR and MALDI-TOF. Subsequently, the antimicrobial activity of oligopeptide fractions was confirmed from assays conducted with various pathogens based on inhibition of microbial growth.

Variation in monomer structure facilitates an ability to use a broad range of natural and non-natural amino acid ester monomers and specific combinations of these monomers as dimers, trimers and higher oligomers to further diversify the peptide material structure.

This invention includes the use of end-group capping methods, such as those described by Viswanathan et al.⁴⁹ as well as other methods that would allow the incorporation of groups, such as azide, alkyne, terminal alkene, furan, norbornene derivatives, and others. Such groups can be incorporated during oligopeptide synthesis or thereafter and can be used to combine polymers of both natural and synthetic origin resulting in the synthesis of hybrid macromolecules with unprecedented properties. Said peptides of this invention may be exploited to induce and control microstructure formation processes in synthetic polymers via bioconjugation. For example, polymers can be organized into tape-like structures using the β-sheet motif, or bundled by packing α-helical peptides of this invention into coiled-coil motifs. Additionally, said peptides based on elastin can be used for tunable temperature responsive cross-linking.

Copolymerization of peptides of this invention may exhibit new biological properties and self-assembly behavior. Additionally, the aforementioned end-groups may be used to conjugate said peptides to various surfaces and substances such as polymer pendant groups and particles of various size and composition. For example, the incorporation of these peptides that have desirable properties, such as intrinsic secondary structures, may be used in the preparation of brush-like polymers using controlled polymerization techniques, such as anionic, radical, and ring-opening metathesis polymerization (ROMP).

Preparation of these polymer bioconjugates can be classified into several main approaches: (i) end-capped said peptides are covalently linked to preformed polymers using one or multiple reactive sites, (ii) synthetic polymer segments are grown in situ from the end-capped said peptides—the macroinitiator strategy, (iii) end-capped said peptides can be used as polymerizable bio-segments in the macromonomer approach.

Said peptides of this invention can also be incorporated as chain segments in natural, synthetic or hybrid polymer chains. Such polymers can be composed exclusively of peptides of this invention; peptide mixtures such as those that include peptides produced as part of this invention and those synthesized by chemical methods, isolated from nature, or produced by recombinant DNA methods; mixtures of peptides produced by methods described in this invention with synthetic or natural non-peptide chain segments. An example of the latter would be to prepare polymers that include chain segments of peptides described herein and polyethylene glycol or other end-group modified forms of polyethylene glycol.

These and other objects, features, and advantages of the present invention will become more apparent to those of ordinary skill in the art when the following detailed description of the preferred embodiments is read in conjunction with the appended figures.

### BRIEF DESCRIPTION OF THE SCHEMES, TABLES, AND FIGURES

Scheme 1 shows the γ-Linkage between glutamic acid and cysteine in phytochelatin analogs (PCs) and α-linkage between glutamic acid and cysteine in co-oligopeptides synthesized by papain-catalyzed oligomerization.

Table 1 shows molecular characteristic of inorganic-binding polypeptide sequences.

Table 2 shows primary sequences or structural motifs leading to peptide amphiphiles, how they self-assemble, and applications of these materials.

Table 3 shows examples of non-natural amino acids or stereochemical mixtures of natural amino acids.

Table 4 shows antimicrobial activity, expressed as %-growth inhibition, by a L-Lys-co-L-Leu oligopeptide^{a} obtained by α-chymotrypsin catalyzed oligomerization of a 1:1 molar feed ratio of L-Lys-Et.2HCl and L-Leu-Et.HCl and subsequent fractionation.

Table 5 shows results of papain (16 units/mL) catalyzed oligomerizations of monomers γ-L-(Et)-2-Glu.HCl and L-Et-Cys.HCl, in 0.9 M sodium phosphate buffer (5 mL) at pH 8.0, 40° C., for 3 h. Total substrate concentration was 0.5 M. Error values are deviations from the mean from at least two experiments.

FIG. 1 shows A) ¹H-NMR spectrum of oligo γ-L-Et-Glu B) ¹H-NMR spectrum of oligo(γ-L-Et-Glu-co-L-Cys) for 4:6 feed ratio of γ-L-(Et)-2-Glu.HCl and L-Et-Cys.HCl C) ¹H-NMR spectrum oligo-L-Cys, synthesized with total monomer concentration of 0.5 M, 16 units/mL papain, 0.9 M phosphate buffer (pH 8.0) at 40° C. for 3 h. The sample was prepared by dissolving ˜10 mg of sample in 25% TFA-d, with TMS as reference.

FIG. 2 shows 2D COSY90 NMR (300 MHz, 25% TFA-d) spectrum of oligo(γ-L-Et-Glu-co-L-Cys)) synthesized using, 7:3 [L-(Et)-2-Glu.HCl:L-Et-Cys.HCl] synthesized with total monomer concentration of 0.5 M, 16 units/mL papain, 0.9 M phosphate buffer (pH 8.0) at 40° C. for 3 h. The sample was prepared by dissolving ˜10 mg of sample in 25% TFA-d, with TMS as reference.

FIG. 3 shows MALDI-TOF mass spectrum of oligo(γ-L-Et-Glu-co-L-Cys)[EₓC_{y}] synthesized as described in the caption for FIG. 1. Asterisk [*] demarcation indicates a peak corresponding to a de-esterified oligomer. Observed values of m/z are ±1 Da of those expected for molecular ion peaks.

FIG. 4 shows absorption spectra of oligo(L-Glu-co-47% L-Cys) at different stages of titration with Cd(II). The reduced peptide was dissolved at a final concentration of 40 μM in 100 mM Tris-HCl (pH 7.41). The dashed line displays results for the oligo(L-Glu-co-47% L-Cys) peptide alone. Spectra were recorded 5 min after addition of Cd(II) as CdSO₄ under anaerobic conditions (by nitrogen saturation at room temperature). Each successive line with increasing absorbance represents the spectrum obtained by the addition of Cd(II) from 0.01 to 6 equivalents. The inset shows the difference of absorbance spectra.

FIG. 5 shows metal ion titration profiles of oligo(L-Glu-co-47% L-Cys) monitored by changes in the absorbance at a specific wavelength. Titrations were conducted at room temperature and pH 7.41 in 100 mM Tris-HCl using four metal ions: (a) ZnSO₄, (b) CdSO₄, (c) CoCl₂, and (d) NiCl₂. The x coordinate is the ratio of the metal ion concentration to the total peptide concentration. The plots represent triplicate experiments. At low [M]ₜₒₜₐₗ/[P]ₜₒₜₐₗ ratios the data were fit by Equation 11 See text for details.

Table 6 shows binding properties of metal complexes. Equilibrium dissociation K_{d} and the product of the molar absorptivity change (Δε) and the path length b are listed.

FIG. 6 shows double log plot for metal ion titration profiles of oligo(L-Glu-co-47% L-Cys) with (a) CoCl₂ and (b) NiCl₂. The change in absorbance was monitored at a specific wavelength at 25° C. and pH 7.4 in 100 mM Tris-HCl. The x coordinate is the ratio of the metal ion concentration to the total peptide concentration. The y coordinate is the difference absorption value after subtracting the contribution of free peptide and free metal ion solution.

FIG. 7 shows the far-UV CD spectra of the oligo(L-Glu-co-47% L-Cys) peptide before (dashed line) and after complexation with (a) Zn(II), (b) Cd(II), (c) Co(II), and (d) Ni(II) complexes (solid lines). The total peptide concentration is 100 μM, and the concentrations of Cd(II), Zn(II), Co(II), and Ni(II) are 400, 400, 1600, and 800 μM, respectively.

FIG. 8 shows Percent Yield and %-Lys incorporation in (L-Lys-co-L-Leu) oligopeptides as a function of reaction pH. Oligomerizations were conducted with total monomer concentration 0.5M using a 1:1 molar feed ratio of L-Lys-Et.2HCl and L-Leu-Et.HCl.

FIG. 9 shows Percent Yield and %-Arg incorporation in (L-Arg-co-L-Leu) oligopeptides as a function of reaction pH. Oligomerizations were conducted with total monomer concentration 0.5M using a 1:1 molar feed ratio of L-Arg-Et.2HCl and L-Leu-Et.HCl.

FIG. 10 shows Percent Yield and %-Lys incorporation in (L-Lys-co-L-Leu) oligopeptides as a function of the comonomer feed ratio. Oligomerizations were conducted with total monomer concentration 0.5M at pH 10.0.

FIG. 11 shows Percent Yield and %-Arg incorporation in (L-Arg-co-L-Leu) oligopeptides as a function of the comonomer feed ratio. Oligomerizations were conducted with total monomer concentration 0.5M at pH 10.0.

FIG. 12 shows MALDI-TOF spectrum of alternating peptide with sequence (AG)ₙ synthesized by α-chymotrypsin at pH 7.

FIG. 13 shows MALDI-TOF spectrum of alternating peptide with sequence (KL)ₙ synthesized by α-chymotrypsin at pH 9.

### DETAILED DESCRIPTION OF PREFERRED EMBODIMENTS

As one example, this invention describes the papain-catalyzed synthesis of cysteine-rich phytochelatin mimetic peptides. By changing the feed ratio of L-cysteine ethyl ester hydrochloride (L-Et-Cys.HCl) and L-glutamic acid diethyl ester hydrochloride [γ-L-(Et)₂-Glu.HCl], papain-catalyzed co-oligomerization produced α-linked oligo-glu/cys peptides that have a ˜1:1 glutamic acid and cysteine residue content with an average chain length of 9. Characterization of the co-oligopeptide was carried out by 1D (¹H) and 2D NMR to estimate the average degree of co-oligopeptide polymerization. MALDI-TOF of co-oligopeptides further revealed that the number of cysteine residues ranged between 2 and 4 per co-oligopeptide. Metal binding properties of co-oligopeptides were studied by UV/vis spectroscopy and CD spectropolarimetry.

Another example illustrating this invention is protease-catalyzed synthesis of lysine-rich and arginine-rich peptides that have useful antimicrobial activity. Proteases useful for the synthesis of lysine-rich and arginine-rich peptides include but are not limited to papain, bromelain, α-chymotrypsin and trypsin. Among these proteases α-chymotrypsin is preferred since it allows the synthesis of relatively longer chain length of co-oligopeptides as determined by ¹H-NMR and MALDI-TOF. By changing the reaction pH and feed ratio of L-lysine ethyl ester dihydrochloride [L-Lys-Et.2HCl] to L-leucine ethyl ester hydrochloride [L-Leu-Et.2HCl], or L-arginine ethyl ester dihydrochloride [L-Arg-Et.2HCl] to L-leucine ethyl ester hydrochloride [L-Leu-Et.2HCl], α-chymotrypsin-catalyzed co-oligomerization produced α-linked oligo(lys-co-leu) and oligo(arg-co-leu) peptides that have about a ˜2:3 molar ratio of Lys/Leu and Arg/Leu and an average chain length by MALDI-TOF analysis of 9. Proton (¹H) NMR was used to determine the average degree of polymerization (DP_{avg}) of co-oligopeptides.

Another example illustrating this invention is fractionation of peptide mixtures to obtain a product mixture with enhanced physical or biological activity. For, example, for oligo(Arg-co-Leu) and oligo(Lys-co-Leu), fractionation can be used to obtain relatively longer chain lengths with higher contents of Lys or Arg, such fractions are expected to have higher antimicrobial activity. Fractionation can be applied to any of the products described in this invention. Methods of fractionation are well known to those skilled in the art. Examples of methods to fractionate peptides in the current invention include but are not limited to: i) differences in solubility in various solvents such as in water with variation in pH, ionicity and concentration of water-miscible cosolvents; ii) differences in affinity to various resins (e.g. ion exchange, reverse phase silica resins, normal phase silica resins) such as those used for chromatography; and iii) separations based on differences in oligopeptide chain length such as by using size exclusion chromatography with size exclusion columns and by using membranes with various molecular weight cutoff value.

Scheme 1 illustrates the difference in structure of PC's that are isolated from nature from both plant and animal sources and the synthetic analogs prepared by protease-catalyzed oligomerizations developed in this invention for use in metal binding.

To mimic the metal binding properties of PC peptides, the efficient protease-catalyzed synthesis of oligo(γ-L-Et-Glu-co-L-Cys) having nearly equal quantities of L-Glu and L-Cys units was performed. By varying the feed ratio of γ-L-(Et)-2-Glu.HCl to L-Et-Cys.HCl, papain-catalyzed oligomerization produced oligo(L-Et-Glu-co-L-Cys) in varying yields and different %-compositions of L-Glu and L-Cys units in precipitated products. Following de-esterification of the products containing about 1:1 L-Glu-to-L-Cys units, the metal binding efficiency was evaluated for divalent metal ions Zn(II), Cd(II), Co(II) and Ni(II).

The molar concentrations of substrates was kept constant (2.5 mmol) while the molar feed ratio of γ-L-(Et)-2-Glu.HCl to L-Et-Cys.HCl was systematically varied from 10:0 to 0:10. The %-yield of co-oligopeptides decreased as the feed ratio of L-Et-Cys.HCl to γ-L-(Et)-2-Glu.HCl increased.

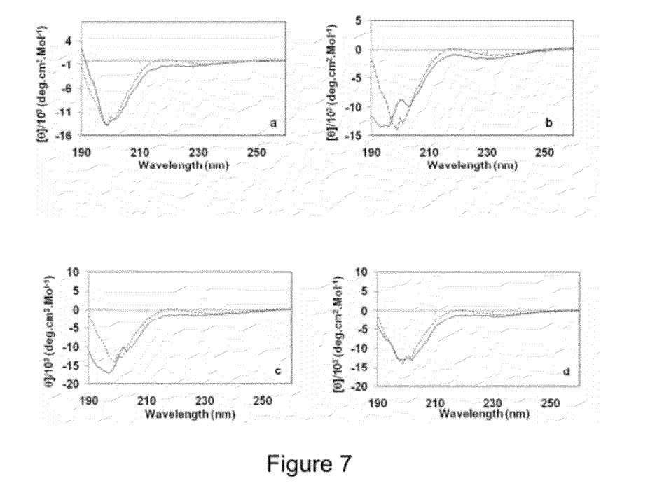

With total substrate molar concentration of 2.5M and a 1:1 molar feed ratio of L-Lys-Et.2HCl-to-L-Leu-Et.HCl 1:1, the reaction pH was systematically varied from 6 to 11. The %-Lys incorporation increases with increasing reaction pH, and the highest %-yield of precipitated co-oligopeptides was obtained at pH 10 (see FIG. 7).

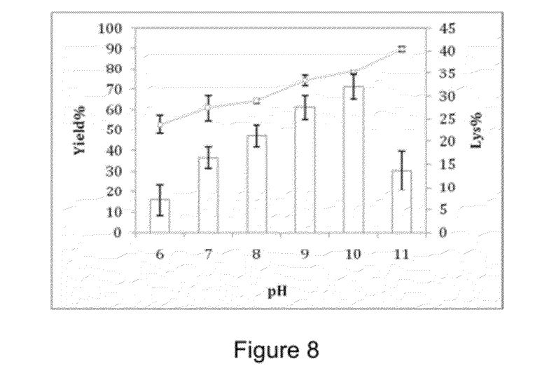

With total substrate molar concentration of 2.5M and a 1:1 molar feed ratio of L-Arg-Et.2HCl-to-L-Leu-Et.HCl 1:1, the reaction pH was systematically varied from 6 to 11. The %-Arg incorporation increases with increasing reaction pH, and the highest %-yield of precipitated co-oligopeptides was obtained at pH 10 (see FIG. 8).

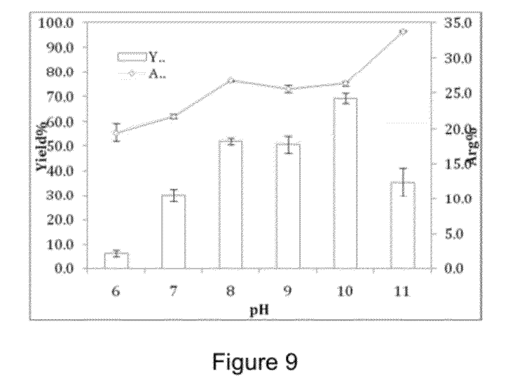

With total substrate molar concentration of 2.5M, pH 10 and variation in the molar feed ratios of L-Lys-Et.2HCl-to-L-Leu-Et.HCl, increase in this feed ratio resulted in a decrease in co-oligopeptide precipitate with increased incorporation of L-Lys (see FIG. 9).

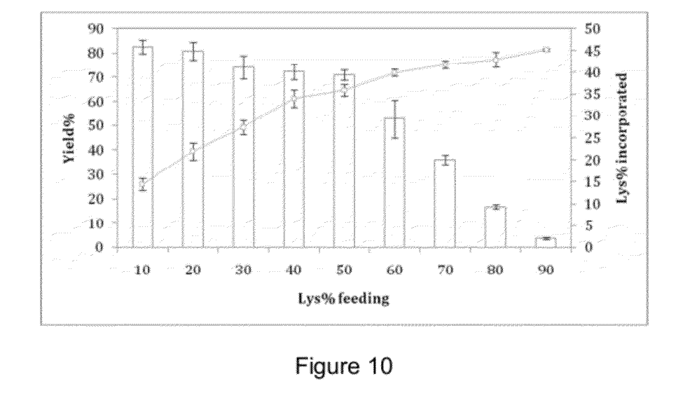

With total substrate molar concentration of 2.5M, pH 10 and variation in the molar feed ratios of L-Arg-Et.2HCl-to-L-Leu-Et.HCl, increase in this feed ratio resulted in a decrease in co-oligopeptide precipitate with increased incorporation of L-Arg (see FIG. 10).

By papain-catalyzed synthesis of oligo(γ-L-Et-Glu-co-L-Cys), up to 4 L-cysteine residues (measured by ¹H-NMR) were incorporated in precipitated product fractions of DP_{avg} 9. The distribution of chain lengths and the distribution of L-cysteine residues an oligopeptide of a specified chain length was evaluated by MALDI-TOF.

The above serve as examples but are not intended to be limiting. There are a wide range of peptide structural motifs that are known to one skilled in the art that provide various types of bioactivity (e.g. antibacterial, antifungal, protein inhibition and antihypertensive, antioxidant, calcium binding, prevent aggregation of platelets, chelate metals or provide other valuable properties). This invention discloses a method by which the beneficial biological and/or physical properties of these peptides are obtained to various extents by incorporating structural motifs of said peptides within peptide mixtures by producing said peptides by protease-catalyzed oligomerization of amino acid alkyl ester monomers and specific combinations of these monomers as dimers, trimers and higher oligomers. Non natural amino acid alkyl esters can be substituted in part or in full for natural amino acid alkyl ester monomers, and in specific combinations of these monomers as dimers, trimers and higher oligomers. Examples of non-natural amino acids are given in the Table 3. Non-natural structures include β-amino acids with D-, L- or a mixture of stereochemical configurations and R-group that are either the same or differ from those found in the naturally occurring 20 amino acids; β-amino acids with D-, L- or a mixture of stereochemical configurations and R-groups that are either the same or differ from those found in the naturally occurring 20 amino acids. Non-natural amino acids also include a larger group of compounds that are well-known to one skilled in the art.

### EXPERIMENTAL

### Materials:

L-Diethyl glutamic acid hydrochloride [γ-L-(Et)₂-Glu.HCl] was purchased from Tokyo Kasei Co. Ltd., L-lysine ethyl ester dihydrochloride [L-Lys-Et.2HCl] was purchased from Alfa Aesar, L-cysteine ethyl ester hydrochloride [L-Et-Cys.HCl], L-arginine ethyl ester dihydrochloride [L-Arg-Et.2HCl] and L-leucine ethyl ester hydrochloride [L-Leu-Et.HCl] were purchased from Aldrich. Thionyl chloride, L-Alanyl-glycine (Ala-Gly) and N_{α},N_{ε}-Di-tert-Butyloxycarbonyl-L-lysine-N-hydroxysuccinimide ester (Boc-Lys(Boc)-OSu) were purchased from Sigma. All the above amino acid ethyl esters were used as received.

Crude papain (cysteine protease EC 3.4.22.2; source, Carcica papaya; 30,000 USP units/mg of solid; molecular weight 21K) was purchased from CalBiochem Co. Ltd. and purified based on a literature protocol.⁵⁰ Bromelain (cysteine protease; EC 3.4.22.4; source, pineapple stem; 2.290 units/mg solid; 3.650 units/mg protein), α-chymotrypsin (serine protease; EC 232-671-2; source, bovine pancreas, type II, 83.9 units/mg solid; 96 units/mg protein), trypsin (serine protease; EC 232-650-8; source, bovine pancreas) were purchased from Sigma Aldrich Inc. and used as received.

Cobalt chloride, zinc sulfate heptahydrate, nickel chloride hexahydrate, cadmium sulfate (anhydrous), deuterated dimethyl sulfoxide (DMSO-d₆), trifluoroacetic acid (CF₃COOH), deuterated trifluoroacetic acid (CF₃COOD) and α-cyano hydroxycinnamic acid (CCA, MALDI-TOF matrix) were purchased from Aldrich and used as received.

Deionized water (DI, 18.2 MΩ·cm purity) was obtained from a RIOS 16/MILLQ Synthesis Millipore water purification system.

### Methods:

Synthesis of Ala-Gly ethyl ester. To a solution of dry cold ethanol (100 mL) containing 1 mmol Ala-Gly was added dropwise 5 mL thionyl chloride at 25° C. with magnetic stirring. After 12 h, volatiles from the solution were removed. The resulting product was dissolved in water and lyophilized to collect the solid product.

Synthesis of Lys-Leu ethyl ester. Into 10 mL DMSO at 25° C. was added Boc-L-Lys(Boc)-OSu (3 mmol), L-Leu ethyl ester (3.3 mmol), and then 0.4 mL triethylamine. The reaction mixture was stirred at room temperature for 2 days and then addition of 1-volume of the reaction mixture to 10-volumes of cold water gave Boc-L-Lys(Boc)-L-Leu ethyl ester as a precipitate. Deprotection was carried out in DCM/TFA (2:1 v/v) for 24 h following a literature procedure⁵¹.

Synthesis of Lys-Phe ethyl ester. Into 10 mL DMSO at 25° C. was added Boc-L-Lys(Boc)-OSu (3 mmol), L-Phe ethyl ester (3.3 mmol), and then 0.4 mL triethylamine. The reaction mixture was stirred at room temperature for 2 days and then addition of 1-volume of the reaction mixture to 10-volumes of cold water gave Boc-L-Lys(Boc)-L-Phe ethyl ester as a precipitate. Deprotection was carried out in DCM/TFA (2:1 v/v) for 24 h following a literature procedure⁵¹.

General procedure for protease-catalyzed synthesis of oligo(X-Y)ₙ alternating peptide, where X and Y represent any hydrophobic amino acid or hydrophobic amino acid derivative, such as Gly, Ala, Val, Leu, Ile, Phe and γ-Et-Glu. Total monomer concentration was fixed at 0.25M in 0.9 M phosphate buffer. After pre-titration by the pH-stat to set reactions at the desired pH, a predetermined amount of protease was added. Reaction solutions were incubated at 40° C. with precise pH control by pH-stat for a predetermined time. The products that precipitate from reaction solutions during reactions were washed twice with 5 mL deionized water and then lyophilized to dry. Resulting oligopeptides were analyzed by NMR and MALDI-TOF to determine composition and chain length distributions.

General procedure for protease-catalyzed synthesis of oligo(Lys-X)ₙ alternating peptide, where X represents any hydrophobic amino acid or hydrophobic amino acid derivative, such as Gly, Ala, Val, Leu, Ile, Phe and γ-Et-Glu. Total monomer concentration was fixed at 0.25M in 0.9 M phosphate buffer. After pre-titration by the pH-stat to set reactions at the desired pH, a predetermined amount of protease was added. Reaction solutions were incubated at 40° C. with precise pH control by pH-stat for a predetermined time. Upon completion of the reaction, protease was removed from the reaction solution by centrifugation using an Amicon Ultra-15 Centrifugal filter with a 3000 molecular weight cut-off membrane. The resulting product was lyophilized for two days to give a white solid. Resulting oligopeptides were analyzed by NMR and MALDI-TOF to determine composition and chain length distributions.

General procedure for protease-catalyzed synthesis of oligo(γ-L-Et-Glu-co-L-Cys) and de-esterified water-soluble oligo(L-Glu-co-L-Cys). The method used was adapted from a literature procedure.⁴⁷ In summary, γ-L-(Et)-2-Glu.HCl (1.25 mmol) and L-Et-Cys.HCl (1.25 mmol) were dissolved in 5 mL of 0.9 M sodium phosphate buffer at pH 8 (optimal pH for synthesis by papain, 16 units/mL),^{47, 52} transferred to a 50 mL borosilicate glass tube, fitted with a Teflon cap and placed in a parallel reactor Carousel 12 (Radleys Discovery Technologies). Reactions were performed with gentle magnetic stirring at 40° C. for 3 h under nitrogen. The reaction mixture was cooled to room temperature, the resulting precipitate was centrifuged and the supernatant was discarded. The precipitate was then washed first with DI water (2×5 mL), then with an HCl solution (pH 2, 2×5 mL). All solutions used for washing were purged with nitrogen for 30 min prior to use, and the remaining solid was lyophilized. Control experiments performed with substrates without addition of enzyme did not yield precipitate. Saponification of precipitated oligo(γ-L-Et-Glu-co-L-Cys) to give fully water-soluble oligo(L-Glu-co-L-Cys) was achieved by reacting the precipitated product with 0.2 M NaOH at 60° C. for 6 h under constant nitrogen bubbling. The clear solution containing the oligo(L-Glu-co-L-Cys) was subsequently neutralized to pH 7 by adding dilute HCl solution and then lyophilized to obtain a beige colored water soluble peptide. Subsequently, the peptide was passed through a Sephadex G-10 desalting column using DI water as eluent and a ninhydrin solution to identify fractions with peptide. All fractions testing positive to ninhydrin were collected and lyophilized to yield purified oligo(L-Glu-co-L-Cys) [EC]₄ peptide. This powder was subsequently used in metal binding assays.

General procedure for protease-catalyzed synthesis of oligo(L-Lys-Et-co-L-Leu-Et). The method used was adapted from a literature procedure.⁴⁷ Total monomer concentration was fixed at 2.5 mmol in 2.5 mL of 0.9 M phosphate buffer. After pre-titration by the pH-stat to set the reaction at the desired pH, 16 units/mL protease (papain, bromelain, α-chymotrypsin and trypsin) was added. Reaction solutions were incubated at 40° C. for 2 h. Precipitated products were washed with 4 mL pH 11 diluted NaOH solution and were then dried (lyophilized one day). Resulting oligopeptides were analyzed by NMR and MALDI-TOF to determine composition and chain length distributions.

General method one to fractionate oligo(L-Lys-Et-co-L-Leu-Et) and oligo(L-Arg-Et-co-L-Leu-Et). After a 2 h reaction to synthesize the oligopeptide, as described above, 20 mL deionized water was added to the reaction mixture. The solution was then titrated to pH 1 by adding 6 M HCl. After centrifuge at 8000 rpm for 8 min, the insoluble precipitate was removed. By adjusting the supernatent to pH 11 at 25° C., the clear solution turned turbid. This cloudy solution is centrifuged at 8000 rpm for 8 min, the insoluble fraction was collected and then lyophilized to give oligopeptide solid powders.

General method two to fractionate oligo(L-Lys-Et-co-L-Leu-Et) and oligo(L-Arg-Et-co-L-Leu-Et). As discussed immediately above, after a 2 h reaction to synthesize the oligopeptide, the reaction mixture was centrifuged at 8000 rpm for 8 min to separate solids and the supernatant. The solids were washed first by methanol (20 mL), then by 2 mL of a pH 12 diluted NaOH solution. The remaining insoluble fraction was collected and lyophilized to obtain oligopeptide solid powders.

Synthesis of α-linked oligo(L-glutamic acid), oligo(L-Glu). The method using papain as catalyst and L-(Et)-2-Glu.HCl as monomer, subsequent hydrolysis of ethyl ester moieties and purification of the product followed exactly as was described previously.^{44,50}

Metal binding assay: Stock solutions of peptide and metal were prepared under strictly anaerobic conditions fulfilled by nitrogen over-saturation (1 h). The concentration of stock [EC]₄ peptide solutions was determined by the 5,5′-dithiobis(2-nitrobenzoic acid), DTNB, assay monitored by UV-visible spectroscopy at 412 nm.⁵³ All buffers were prepared using metal free reagents and deionized water. The metal binding affinity of each metal ion was measured by spectrophotometric titration of the metal stock solution into a 100 mM tris(hydroxymethyl)aminomethane (Tris.HCl) buffer solution (pH 7.4) containing a predetermined concentration of the peptide (˜40 μM).

Antimicrobial activity assay: Antimicrobial assay was performed using 96 well micro-plates with 11 human pathogens including 10 bacteria and 1 yeast culture. Bacteria used were Proteus milrabillis, Staphylococcus aureus, Pseudomonas aeruginosa, Bacillus subtilis, Escherichia coli, Klebsilla pneumonia, Nesseria gonoria, Morexella sp., Proteus vulgaris, Streptococcus neumonia. The yeast used was Candida tropicalis. Peptide stock solutions (100 μL) were placed in the first row of wells in the micro-plate and serially diluted using Brain Heart Infusion Broth (BHIB). An 80 μL aliquot of fresh culture medium was transferred into the wells having 100 μL of diluted peptide solution to bring the total volume to 180 μL. Microbial cultures were grown in BHIB for 18 h at 37° C. and diluted using the same fresh culture medium to obtain an optical density (OD) of 0.5 at 540 nm. A 20 μL aliquot of diluted bacterial and yeast cultures (with optical density of ˜0.5) were added in to the above wells with serially diluted peptide solution and incubated for 24 h at 37° C. In order to determine the growth of cultures without peptide, control cultures were conducted by replacing 100 μL peptide solutions with 100 μL culture broth (100 μL). Also, the antibiotic streptomycin was used as a standard to compare the antimicrobial activity of peptides. Antimicrobial activity was estimated by measuring the OD of the above micro-plates with microbial culture and peptide solution at 540 nm. OD measured immediately after inoculation of the bacterial culture was taken as 0 h. Subsequently, OD measurements were recorded at 12 and 24 h after incubation. OD obtained in control cultures without peptide was compared with OD observed with peptides, the difference in the OD was converted into percentage (%) and the antimicrobial activity was expressed in % of growth inhibition. Minimum Inhibitory Concentration (MIC) was determined from the above results and is defined as the peptide concentration that inhibits microbial growth by more than 95%.

### Instrumental Methods:

Nuclear Magnetic Resonance (NMR) spectroscopy: Proton (¹H) NMR spectra were recorded on a Bruker DPX 300 spectrometer at 300 MHz. Oligo(γ-L-Et-Glu-co-L-Cys) products (10 mg/mL) were dissolved in a 1:3 mixture of CF₃COOD/CF₃COOH or a 1:9 mixture of D₂O/H₂O. Oligopeptide products consisting of L-Lys-Et-co-L-Leu-Et) and oligo(L-Arg-Et-co-L-Leu-Et) were dissolved (10 mg/mL) in a 30:1 mixture of DMSO-d₆/CF₃COOH. A total of 128 scans were collected and analyzed by MestRec-C software. Proton chemical shifts were referenced to tetramethylsilane (TMS) or 4,4-dimethyl-4-silapentane-1-sulfonic acid (DSS) at 0.00 ppm in CF₃COOD and D₂O, respectively.

Matrix-assisted-laser-desorption/ionization Time-of-flight (MALDI-TOF) mass spectroscopy: MALDI-TOF spectra were obtained on an OmniFlex MALDI-TOF mass spectrometer (Bruker Daltonics, Inc.). The instrument was operated in a positive ion reflector mode with an accelerating potential of +20 kV. The linear flight path is 120 cm. OMNIFLEX TOF control software was used for hardware control and calibration. Spectra were obtained by averaging at least 300 laser shots. The pulsed ion extraction delay time was 200 ns. The spectrometer was calibrated using Angiotensin II as the external standard (1046.54 amu). To generate the matrix, a saturated solution of α-cyano-4-hydroxycinnamic acid (CCA) was prepared in a water-acetonitrile mixture (2:1 v/v) with 0.1% TFA (TA solution).⁵⁴ A stock solution of oligo(γ-L-Et-Glu-co-L-Cys) was prepared by dissolving ˜1 mg of the peptide in 200 μL DMSO with 0.1% TFA containing 2 equivalents (with respect to thiol group content) D,L-dithiothreitol as reducing agent. Also, a stock solution of oligo(L-Lys-co-L-Leu) or oligo(L-Arg-co-L-Leu) was prepared by dissolving ˜1 mg of the peptide in 200 μL DMSO with 1% TFA or in 200 μL 1,1,1,3,3,3-hexafluoro-2-propanol (HFIP). The stock solution (10 μL) was further diluted with 240 μL of TA solution so that the final concentration of oligopeptide was ˜40 pmol/μL. A 10 μL aliquot of this solution was mixed with 10 μL of CCA (matrix) solution in a 100 μL Eppendorf tube. Then, 0.5 μL of this mixture was applied to the steel target and dried under vacuum. Experimentally determined masses, obtained using XMASS-OMNIFLEX6.0.0 software, were compared to a theoretical database created in MS Excel for oligo(γ-L-Et-Gluₓ-co-L-Cys_{y})ₙ peptide where n=x+y; x=0-12, y=0-12.

UV-visible spectroscopy: UV-visible spectra were recorded on a LAMBDA 800 Perkin Elmer UV/VIS spectrometer using quartz cells with open top screw caps fitted with septa. Transfer of solutions under anaerobic conditions was carried out using a gas-tight Hamilton syringe. Wavelength scans were performed at room temperature, in the range of 200-600 nm, at a scan speed of 2600 nm/min with 10 nm intervals. Determination that equilibrium was reached at each step of the titration was based on that no further change in absorbance was observed (typically 5 min).

### Results

To mimic the structure of PC peptides, studies were carried out to synthesize oligo(γ-L-Et-Glu-co-L-Cys) having equal molar quantities of L-Glu and L-Cys units. By varying the feed ratio of γ-L-(Et)₂-Glu.HCl to L-Et-Cys.HCl, papain-catalyzed oligomerization produced oligo(L-Et-Glu-co-L-Cys) in varying yields and with different contents of L-Glu and L-Cys units. Following de-esterification of the product containing ˜1:1 L-Glu and L-Cys units, the metal binding efficiency for divalent metal ions Zn(II), Cd(II), Co(II) and Ni(II) was evaluated.

Effect of the monomer feed ratio on the %-composition of oligo(L-Et-Glu-co-L-Cys): The molar concentrations of total substrates in oligomerization reactions was kept constant (2.5 mmol), and the feed ratio of L-(Et)₂-Glu.HCl to L-Et-Cys.HCl was systematically varied from 10:0 to 0:10. Table 5 lists values of oligopeptide %-yield and composition as a function of the monomer feed. The %-yield of co-oligopeptides decreased from 70±6 to 44±5 by increasing the content of L-Et-Cys.HCl in the monomer feed from 0 to 30 mol %. Further increases of L-Et-Cys.HCl to 50 and 100% of the monomer feed resulted in further decreases in %-yield to 40±4 and 25±7, respectively. This result is explained by that, increased L-cysteine incorporation into the growing oligopeptide chain results in increased oligopeptide water solubility and, hence, a lower yield of precipitated oligo(γ-L-Et-Glu-co-L-Cys). Analysis of oligo(γ-L-Et-Glu-co-L-Cys) average chain length (DP_{avg}), average composition of Glu and Cys units, and chain length distribution was performed by ¹H-NMR and MALDI-TOF as discussed herein.

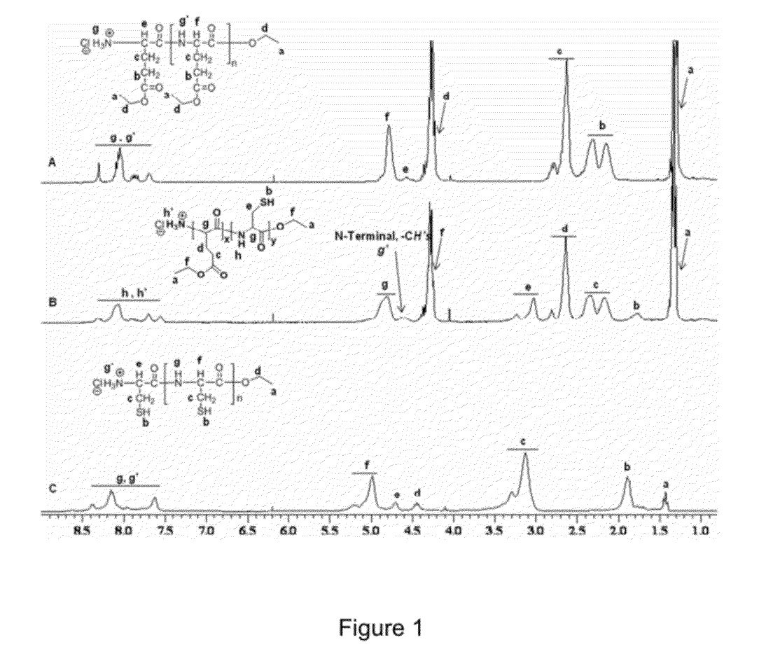

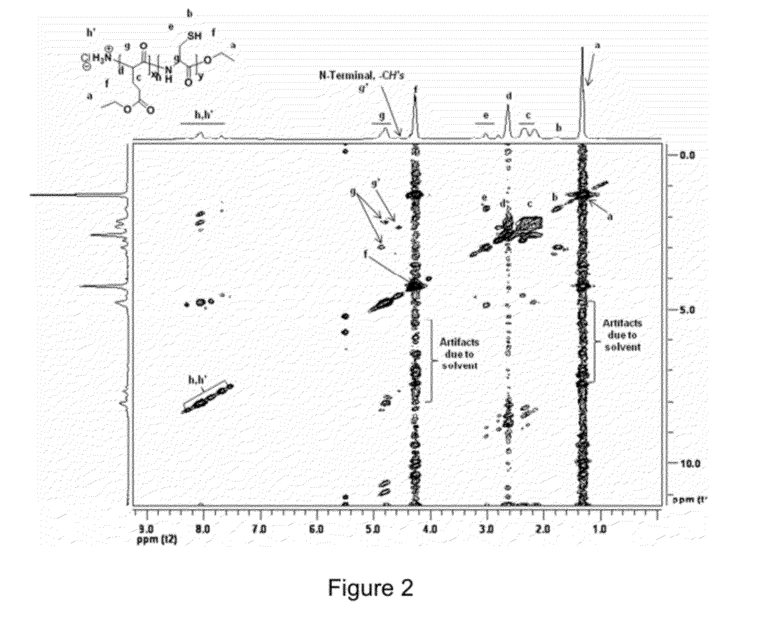

Structural analysis by ¹H-NMR Spectroscopy: The spectrum of papain-catalyzed oligo(γ-L-Et-Glu) was recorded in DMSO-d₆. Peak assignments and corresponding DP_{avg} calculations were based on previously published methods^{47, 49, 55}. Analysis of oligo(γ-L-Et-Glu-co-L-Cys) was performed in 25% TFA-d, with TMS as reference (FIG. 1). Methine resonances of γ-L-Et-Glu repeat units (including those at the C-terminus) are found at 4.7-5 ppm (protons g), whereas methine resonances of N-terminal γ-L-Et-Glu and L-Cys units are at 4.53-4.68 ppm (protons g′). These assignments were made based on analysis of the corresponding 2D NMR (¹H-¹H COSY90) spectrum (see FIG. 2). C-Terminal methine and main chain methine protons g (—CH—COO[H/Et]) at 4.7-5 ppm correlated to proton h (—OC—NH—CH—COO[H/Et]; —HN—CH—CO—) at 7.8, 8.0, 8.25 ppm and methylene protons c, d, e (—CH₂CH₂—COOEt; —CH₂—SH) at 2.2-2.3, 2.6-2.8 and 3.0 respectively, as well as to proton b (—CH₂SH) at 1.75 ppm (See FIGS. 1C, 2). The N-terminal methine protons g′ (H₃N—CH—CO—) at 4.53-4.68 ppm of γ-L-Et-Glu and L-Cys units correlated to —NH₃ (h′) protons at 7.5-7.67 ppm and methylene protons c, d, e, b (—CH₂CH₂—COOEt and —CH₂—SH) at 2.36, 2.65, 3.18 and 1.78 ppm, respectively. The DP_{avg} was determined from the relative peak intensities of N-terminal methine protons g′ and internal methine protons g. Signals corresponding proton c (—CH₂CH₂—COOEt) at 2.2-2.3 ppm and e (—CH₂—SH) at 3.18 ppm were used to determine the co-oligomer composition. Other peaks at 4.33 and 1.37 ppm correspond to ethyl ester protons f and a, respectively (—COOCH₂CH₃), correlate to each other.

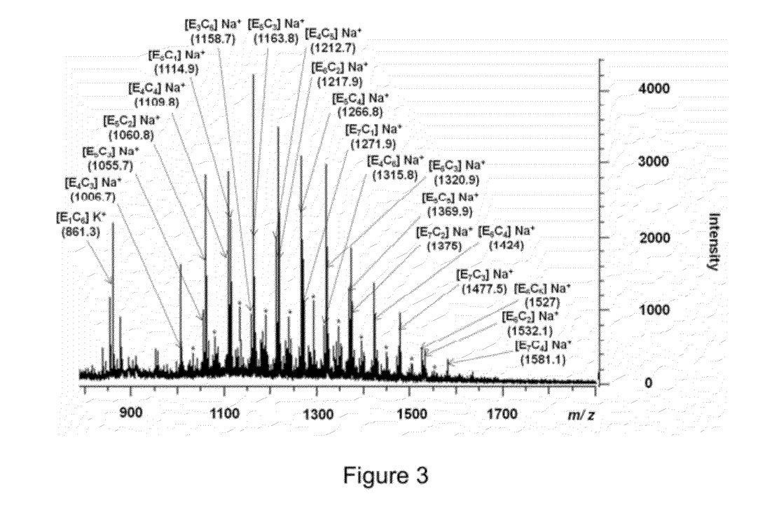

For the MALDI-TOF analysis of oligo(γ-L-Et-Glu-co-L-Cys), this oligopeptide is abbreviated [EₓC_{y}], where E is the γ-Et-ester, in order to facilitate labeling and discussion of peaks in MALDI-TOF spectra. MALDI-TOF spectra obtained for different feed ratios of γ-L-(Et)₂-Glu.HCl-to-L-Et-Cys.HCl, produced a series of signals in MALDI-TOF spectra corresponding to [EₓC_{y}]. For example, the MALDI-TOF spectrum for the oligopeptide synthesized using a 4:6 feed ratio of γ-L-(Et)₂-Glu.HCl-to-L-Et-Cys.HCl, is shown in FIG. 3. The peaks at m/z=861.3 to 1581.1 correspond to E₁C₈ and E₇C₄, respectively, associated with Na⁺. Further analysis of the MALDI-TOF spectrum reveals the most abundant signals correspond to co-oligopeptides with DP 9, in excellent agreement with DP_{avg} 9.2, determined by ¹H-NMR spectroscopy (Table 5). Among all repeat unit combinations with DP 9, highest peaks in the series correspond to the co-oligopeptides with repeat unit compositions E₄C₅ (1212.7; [MI+Na⁺]), E₅C₄, (1266.8; [MI+Na⁺]), E₆C₃ (1320.9; [MI+Na⁺]), and E₇C₂ (1375; [MI+Na⁺]). Similarly, for DP 8, co-oligopeptides E₄C₄ (1109.8; [MI+Na⁺]), E₅C₃ (1212.7; [MI+Na⁺]), and E₇C₁ (1271.9; [MI+Na⁺]) give peaks of highest intensities. This distribution indicates that papain produced a mixture of peptides that favor addition of γ-L-(Et)-2-Glu.HCl over L-Et-Cys.HCl. However, this data is misleading since oligopeptides with higher contents of Cys units are more soluble and will tend to remain in the reaction media. As discussed above, analysis of oligopeptides produced herein was restricted to products that precipitate from the reaction medium. Previous work by our laboratory by Li et al.⁴⁷ for papain-catalyzed co-oligomerization of L-Glu-(Et)₂ and L-Leu-Et showed that papain showed no apparent selectivity for addition of either co-monomer. Hence little or no selectivity for papain-catalyzed oligomerizations of γ-L-(Et)-2-Glu.HCl over L-Et-Cys.HCl would not be unexpected nor extraordinary. For feed ratios of 7:3, 6:4 and 5:5 γ-L-(Et)-2-Glu.HCl-to-L-Et-Cys.HCl, oligo(γ-L-Et-Glu) peaks corresponding to E₅, E₆, and E₇, associated with one Na⁺, were observed. However, for feed ratios of 4:6 and 3:7 γ-L-(Et)₂-Glu.HCl-to-L-Et-Cys.HCl, peaks corresponding to homo-oligomers of glutamate ester units were not observed. Correspondingly, co-oligomers formed had relatively higher incorporation of cysteine units. This is consistent with composition data from ¹H-NMR (Table 5) that shows by increasing the feed ratio of γ-L-(Et)-2-Glu.HCl-to-L-Et-Cys.HCl from 7:3 to 5:5 and 3:7, the mol %-Cys in co-oligomers increased from 25±2 to 37±3 to 55±5. Furthermore, the MALDI-TOF analysis of peaks showed overlap of theoretical molecular weights of two different sequences, the authentication of peaks and subsequent unambiguous peak assignments were performed by using the Isotope pattern calculator (IPC) program that is available from PNNL and can be downloaded from OMNICS.PNL.GOV.

Reactivity ratios were determined by the Mayo-Lewis slope-intersection method as is described elsewhere⁴⁷. Values for γ-L-(Et)₂-Glu.HCl and L-Et-Cys.HCl are 0.70 and 0.45, respectively, indicating that synthesized oligopeptides tend towards a random sequence distribution. It is understood by one skilled in the art that by changing the protease-catalyst used co-oligopeptides can be obtained that can approach to various degrees random, block and alternating-type sequences. Furthermore, by using a dipeptide such as [γ-L-(Et)-Glu]-[L-Et-Cys.HCl], alternating type oligopeptides will be formed.

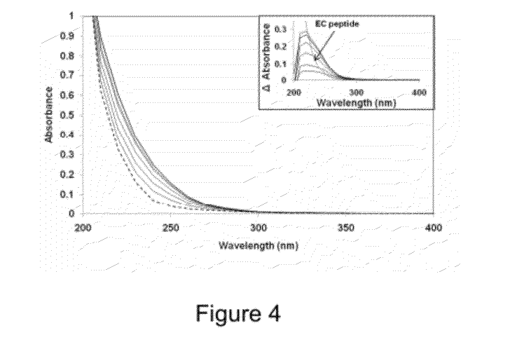

Metal binding assay: For metal binding studies, oligo(L-Glu-co-47% L-Cys) containing 47±4% Cys units, obtained by using a 4:6 ratio of γ-L-(Et)-2-Glu.HCl-to-L-Et-Cys.HCl in the monomer feed (Table 5), was selected since its composition most closely matched that of PC's (50:50 Glu-to-Cys). Experiments were performed to determine the metal binding properties for this mixture of peptides and the results were compared to that obtained by Cheng et al.³ for a uniform sequence of [γ-Glu-Cys]₄-Gly peptide synthesized by solid phase peptide synthesis. All titration experiments were performed in triplicate. FIG. 4 shows that, addition of Cd(II) to a 40 μM solution of oligo(L-Glu-co-47% L-Cys) increases the absorbance in the 230 to 260 nm range. The observed difference spectrum at about 250 nm (FIG. 4 inset) is attributed to ligand-to-metal charge transfer (LMCT) bands.^{3, 56-58} The absorption by Cd(II) in this wavelength range is negligible.

Likewise, titration experiments were performed in triplicate with Zn(II), Co(II) and Ni(II). The binding efficiency of oligo(L-Glu-co-47% L-Cys) with each of these metals was performed by determining the absorbance difference at the wavelength specific to its LMCT band (i.e. Zn(II) 230 nm, Cd(II) 250 nm, Co(II) 250 nm, Ni(II) 260 nm).

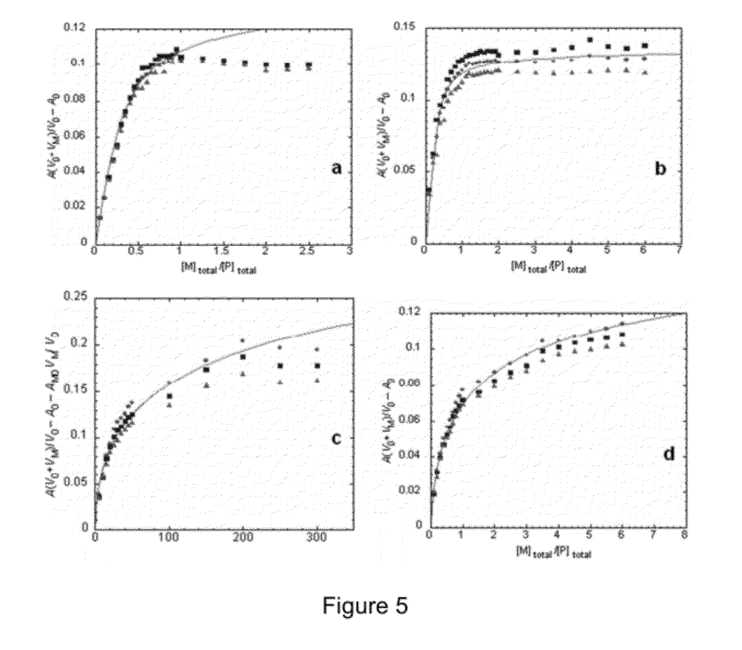

For quantitative analysis, the absorbance at each stage of titration was corrected for dilution by the volume increase and the difference in absorbance was calculated as ΔA. The concentration of the peptide was estimated assuming it has a uniform chain length of 9 units and an average of four cysteine residues per peptide chain (from ¹H NMR analysis, Table 5). FIG. 5 shows ΔA as a function of the ratio of total metal ion concentration to total peptide concentration. The four plots in FIG. 5 are for titrations with Zn(II) (5a), Cd(II) (5b), Co(II) (5c), and Ni(II) (5d). The general trend for plots in FIG. 5 is ΔA increased and approached a plateau or maximum value as more metal ions were added. Inspection of FIG. 5c shows that Co(II) required a total metal-to-peptide molar ratio ([M]ₜₒₜₐₗ/[P]ₜₒₜₐₗ) that is about two orders of magnitude higher than that required for titrations with Zn(II), Cd(II) and Ni(II) to reach a plateau or maximum value of ΔA.

γ-Linked peptides of uniform sequence and length that mimic PCs also have been synthesized but by using conventional solid-state methods. Mehra⁵⁷ prepared the pentapeptide [γ-Glu-Cys]₂-Gly with γ-linked Glu units and analyzed the peptides binding to Cd(II) by the method described herein. Titration with increasing amounts of Cd(II) induced the formation of a pronounced shoulder at 240 nm. Plot of ΔA vs. [M]ₜₒₜₐₗ/[P]ₜₒₜₐₗ showed that the increase in ΔA flattened upon saturation of Cd(II) binding capacity. In fact, Mehra reported that a small decrease in absorbance at 240 nm was observed once binding of Cd(II) to [γ-Glu-Cys]₂-Gly saturated at 0.5 equivalents.⁵⁷ Similarly Kobayashi and Yoshimura⁵⁸ observed an enhancement in absorption intensity at 250 nm for Cd(II) titration to [γ-Glu-Cys]₂-Gly up to a 1.4 ratio of Cd(II)/[γ-Glu-Cys]₂-Gly. Above this ratio they observed a gradual decrease in intensity.⁵⁸ Study of spectroscopic properties of Cd(II), Ni(II) and Co(II)-bound wild-type and non-native retroviral-type zinc finger peptides by Chen et al.⁵⁹ showed a similar plateau effect with increase in Co(II) concentration for ΔA at 650 nm.

### Peptide-Cation Complex and Photometric Titration

In general, a peptide-cation complex consists of a peptide molecule (P) and n cations (M), where n may not be an integer. In the equilibrium

PMₙ [symbol] P+nM (1)

the dissociation constant K_{d} of the complex (PMₙ) is expressed as

$${K}_{d} = \frac{{[ P ] [ M ]}^{n}}{[ {PM}_{n} ]} \qquad ( 2 )$$

Before adding cations, the absorbance A₀ of the peptide solution of concentration [P]₀ is given as

$$\frac{{A}_{0}}{b} = {{ɛ}_{P} [ P ]}_{0} \qquad ( 3 )$$

where b is the path length of the cell, and ε_{P} is the molar absorptivity of the peptide. If the absorbance by cations is negligible, the absorbance A of a titrated solution that has complexed peptide at concentration [PMₙ] and uncomplexed peptide at concentration [P] is given as

$$\frac{A}{b} = {ɛ}_{PM} [ {PM}_{n} ] + {ɛ}_{P} [ P ] \qquad ( 4 )$$

where ε_{PM} is the molar absorptivity of the complexed peptide.

In titration experiments, the total concentrations of P and M ([P]ₜₒₜₐₗ, [M]ₜₒₜₐₗ) are specified, rather than uncomplexed species P and M. The [P]ₜₒₜₐₗ and [M]ₜₒₜₐₗ after volume V_{M} of titrant of concentration [M]₀ is added to the peptide solution of volume V₀ are

$$\begin{matrix}{[ P ]}_{total} = [ P ] + [ {PM}_{n} ] = {[ P ]}_{0} \frac{{V}_{0}}{{V}_{0} + {V}_{M}} & ( 5 ) \\ {[ M ]}_{total} = [ M ] + n [ {PM}_{n} ] = {[ M ]}_{0} \frac{{V}_{M}}{{V}_{0} + {V}_{M}} & ( 6 )\end{matrix}$$

The absorbance difference ΔA is calculated for the titrated solution, corrected for dilution:

$$Δ A ≡ A \frac{{V}_{0} + {V}_{M}}{{V}_{0}} - {A}_{0} = {b ( {ɛ}_{PM} - {ɛ}_{P} ) [ {PM}_{n} ]}_{0} \qquad ( 7 )$$

where [PMₙ]₀ is the concentration of the complex peptide were it not for the dilution:

[PMₙ]₀=[PMₙ](V₀+V_{M})/V₀.

If the absorbance by cations is not negligible (molar absorptivity is ε_{M}), the expression for ΔA needs an additional correction:

$$Δ A ≡ A \frac{{V}_{0} + {V}_{M}}{{V}_{0}} - {A}_{0} - {A}_{M 0} \frac{{V}_{M}}{{V}_{0}} = {b ( {ɛ}_{PM} - {ɛ}_{P} - n {ɛ}_{M} ) [ {PM}_{n} ]}_{0} \qquad ( 8 )$$

where A_{M0} is the absorbance of the titrant.

### Discussion of Titration Curves

The titration curves for ZnSO₄ and CdSO₄ in FIGS. 5a and 5b have an initial steep rise, followed by a rapid transition to a plateau level. This dependence on [M]ₜₒₜₐₗ/[P]ₜₒₜₐₗ indicates strong binding, and can be analyzed as follows. At low concentrations of cations, nearly all cationic species bind to the peptides: [M]ₜₒₜₐₗ≈n[PMₙ]. Then, [PMₙ]≈([M]ₜₒₜₐₗ/[P]ₜₒₜₐₗ)([P]ₜₒₜₐₗ/n). Conversion of [PMₙ] to [PMₙ]₀ and [P]ₜₒₜₐₗ to [P]₀ gives

$${[ {PM}_{n} ]}_{0} ≅ \frac{{[ M ]}_{total}}{{[ P ]}_{total}} \frac{{[ P ]}_{0}}{n} \qquad ( 9 )$$

At high concentrations of cations, nearly all peptides are complexed: [PMₙ]≈[P]ₜₒₜₐₗ, i.e., [PMₙ]₀≈[P]₀. Since ΔA is proportional to [PMₙ]₀, FIG. 4 is essentially a plot of [PMₙ]₀. The plot of [PMₙ]₀ as a function of [M]ₜₒₜₐₗ/[P]ₜₒₜₐₗ will be a straight line with a slope [P]₀/n at low [M] and quickly changes to a horizontal line at [P]₀. The two asymptotes intersect at [M]ₜₒₜₐₗ/[P]ₜₒₜₐₗ=n. In the titrations with ZnSO₄ and CdSO₄, FIGS. 5a and 5b, the crossover is seen at around [M]ₜₒₜₐₗ/[P]ₜₒₜₐₗ=0.5. Thus we find that n=0.5 for complexation, and the complexes are PM_{1/2} or P₂M, that is two peptide molecules per divalent cation.

In contrast, the rise in the titration curves for CoCl₂ and NiCl₂ in FIGS. 5c and 5d is slow, indicating weak binding. At low concentrations of cations, most of the cations and peptide molecules are not complexed. Therefore, equation 2 reduces to

$${K}_{d} ≅ \frac{{[ P ]}_{total} {( {[ M ]}_{total} )}^{n}}{[ {PM}_{n} ]} \qquad ( 10 )$$

When V_{M}<<V₀,

$${[ {PM}_{n} ]}_{0} ≅ {( \frac{{[ M ]}_{total}}{{[ P ]}_{total}} )}^{n} \frac{{( {[ P ]}_{0} )}^{n + 1}}{{K}_{d}} \qquad ( 11 )$$

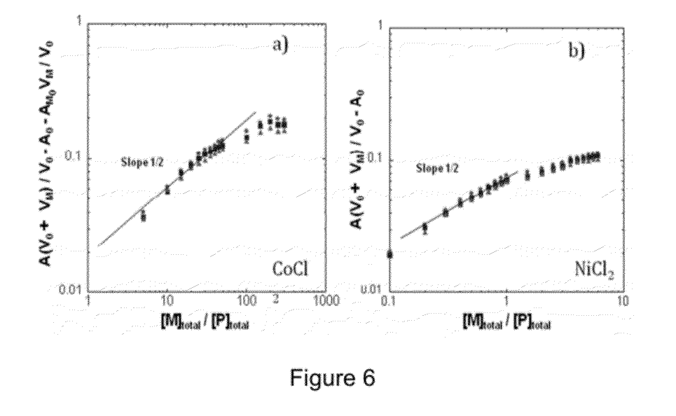

Therefore, ΔA, plotted as a function of [M]ₜₒₜₐₗ/[P]ₜₒₜₐₗ in double-log scale, will follow a straight line of slope n. FIGS. 6a and 6b show such plots. The data at low concentrations lie along a straight line with a slope of 0.5. For Ni(II) and Co(II) cations, formation of the complex P₂M is likely.

Since n=0.5 is indicated for all four of the cations, we fit titration curves assuming formation of a P₂M complex by combining equations 5 and 6 to give equation 12.

$${[ M ]}_{total} = \frac{1}{2} [ {PM}_{n} ] + {( \frac{{K}_{d}}{{[ P ]}_{total} / [ {PM}_{n} ] - 1} )}^{2} \qquad ( 12 )$$

where [P]ₜₒₜₐₗ/[PMₙ]=[P]₀/[PMₙ]₀. Then, equation 12 is further rewritten to

$${[ M ]}_{total} = \frac{{n [ {PM}_{n} ]}_{0} + {( \frac{{K}_{d}}{{[ P ]}_{0} / {[ {PM}_{n} ]}_{0} - 1} )}^{1 / n}}{1 + {n [ {PM}_{n} ]}_{0} / {[ M ]}_{0}} \qquad ( 13 )$$

In this equation, [P]₀ and [M]₀ are fixed. Since ΔA is proportional to [PMₙ]₀, equation 11 was used to fit data in the plot of [M]ₜₒₜₐₗ as a function of ΔA. The solid lines in FIGS. 5a through 5d are the results of the fitting. For plots in 5b and 5c, the whole range of the data was used for the fitting, but for plots 5a and 5d, the ranges used are [M]ₜₒₜₐₗ≦4.5×10⁻⁵ and [M]ₜₒₜₐₗ≦0.006896 M. However, the last 2 points in the experimental data closest to 0.006896 M were not included in the calculation of the theoretical curve fitting equation. Table 6 lists values from curve fitting of K_{d} and molar absorptivity change Δε (ε_{PM}−ε_{P}) for Zn(II), Ni(II) and Cd(II) as well as ε_{PM}−ε_{P}−0.5ε_{M} for Co[II]. With ε_{P}=705 L·mol⁻¹cm⁻¹, 950 L·mol⁻¹cm⁻¹ and 3692.5 L·mol⁻¹cm⁻¹ at 260, 250 and 230 nm, respectively, and ε_{M 260nm}=12.75 L·mol⁻¹cm⁻¹ [Co(II)], estimation of ε_{PM} for the four complexes was performed and the results are listed in Table 6. Based on the dissociation constant, the binding efficiency of the metals to oligo(L-Glu-co-47% L-Cys) follows the order Cd>Zn>Ni>Co. In case of [(γ-EC)₄]-Gly peptides synthesized by Cheng et al.³ the order was similar (Cd>Zn>Ni>Co). Thus, protease-catalysis of Glu-co-Cys peptides gave us distinctive peptide mixtures with metal binding properties that mimic that of peptides synthesized by tedious step-by-step chemical peptide synthetic methods. Such a result could not be anticipated based on other information in the literature.

Furthermore, the peculiar behavior where ΔA decreases at [M]ₜₒₜₐₗ/[P]ₜₒₜₐₗ>1 for Zn(II) titration cannot be described by equation 13. A similar behavior was observed in past studies for alternating co-oligopeptides and random co-oligopeptides containing cysteine synthesized by solid phase chemical methods.^{60,57}

The decrease may be attributed to the diversity of structures that are comprised within oligo(L-Glu-co-47% L-Cys). That is, oligo(L-Glu-co-47% L-Cys) consists of a mixture of peptides that differ in sequence as well as chain length. Work by Opella et al.¹ provides an example that supports this explanation. These workers evaluated the metal binding of three 18 residue-peptides with different sequences of cysteine, i.e., CAAC, CACA and CCAA. They found that only peptides with vicinal cysteines bind mercury.¹ Furthermore Luczkowski et al.⁵⁶ synthesized cysteine derivatives of the trimeric (TRI) peptide family (Ac-G(LKALEEK)₄G-NH₂), known to bind heavy metals in an unusual trigonal geometry. On titrating Cd(II) into a buffered solution of —Cysₐ-Xaa-Xaa-Cys_{d}-(TRIL9CL12C) peptide, where Xaa represents any of the 20 naturally occurring amino acids, they observed linear binding behavior until [M]ₜₒₜₐₗ/[P]ₜₒₜₐₗ reached 0.3. Further addition of Cd(II) formed a different complex having 0.6 equivalents of metal ion per peptide.⁵⁶ The decrease in ΔA observed herein is also ascribed to formation of a different complex at [M]ₜₒₜₐₗ/[P]ₜₒₜₐₗ>1 that disrupts the structure formed at lower M-to-P ratios.

As a control experiment, oligo-(γ-Et-L-Glu) was synthesized from L-(Et)-2-Glu using papain as catalyst⁵⁰ and de-esterified by hydrolysis. Spectroscopic titration was performed on the corresponding fully water-soluble oligo(Glu) sample. The concentrations of the peptide and metal ions were similar to those used for evaluating oligo(L-Glu-co-47% L-Cys) metal binding profiles. No significant change in the UV-visible spectra was observed for Zn(II), Cd(II), and Ni(II) at [M]ₜₒₜₐₗ/[P]ₜₒₜₐₗ ratios up to 1. However, an increase in absorption was observed for Zn(II) and Co(II) titrations at [M]ₜₒₜₐₗ/[P]ₜₒₜₐₗ ratios >1. This is believed to be a consequence of interaction of the metals with γ-carboxyl and terminal-NH₂ moieties.

In the case of Co(II) titration of oligo(Glu), increase in concentration of Co(II) by itself gives minor contribution to the net absorption. Thus, we conclude that the change in absorption observed for oligo(L-Glu-co-47% L-Cys) with DP_{avg} 9 prepared by protease catalysis mimics the behavior of PC peptides by sharing the phenomena of thiol-mediated coordination to different divalent metals.

The CD spectrum of oligo(L-Glu-co-47% L-Cys) peptide in aqueous solution exhibits strong negative bands at 198 and 202 nm, indicating a random coil conformation.⁶¹ A similar CD profile was reported for a uniform sequence of (yGlu-Cys)₄-Gly peptide.³ On coordination of oligo(L-Glu-co-47% L-Cys) by Zn(II) or Ni(II), small changes in the CD spectrum consisting of decreases in the minima at 196 nm and 203 nm were observed. For Co(II) complexes, a marked decrease in the minima at 198 nm was observed. These changes are attributed to the higher metal ion concentration used relative to the above two metals. That is, for total peptide concentration of 100 μM, concentrations of Zn(II), Cd(II) and Ni(II) are 400, 400 and 800 M whereas the concentration of Co(II) is 1600 M.

In the case of (γGlu-Cys)-4-Gly peptide, Cheng et al.³ reported CD spectra with double negative minima at 203 nm and 230 nm for Cd(II) complexes, which they attributed to folding of the peptide into a turned conformation such as an 3₁₀ helix turns or β-turns. One would expect similar phenomena for oligo(L-Glu-co-47% L-Cys) and related peptide compositions prepared by protease catalysis based on the UV-visible data discussed in the previous section. Since the net change in CD is an average of contributions from the mixture of peptides present in oligo(L-Glu-co-47% L-Cys), the structural changes associated with metal binding to oligo(L-Glu-co-47% L-Cys) is further diluted. Analogous experiments with oligo(Glu), synthesized by papain catalysis, were performed to further validate if the small changes observed in CD for oligo(L-Glu-co-47% L-Cys) peptide on metal ion reconstitution were indeed due to thiolate chelation of metal ions. The CD spectra observed for oligo(Glu) titrated with all four metals used above did not show significant changes, validating that the small changes observed for oligo(L-Glu-co-47% L-Cys) are indeed due to a thiolate mediated chelation effect.

FIG. 8 shows results of %-yield and %-Lys incorporation in (L-Lys-co-L-Leu) oligopeptides as a function of reaction pH. Oligomerizations were conducted with total monomer concentration 0.5M using a 1:1 molar feed ratio of L-Lys-Et.2HCl and L-Leu-Et.HCl. The product formed precipitates from the reaction during oligomer synthesis. Study of FIG. 8 shows that, maximum % yield of oligo(L-Lys-co-L-Leu) was obtained at pH 10, whereas, maximum %-Lys incorporation occurred at pH 11. With increasing pH from 6 to 10, %-yield increases from 16±8 to 71±6, while %-Lys content increased from 24±2 to 35±1. However, as the pH increases from pH 10 to 11, %-Lys increases from 35±1 to 40±1 while %-yield decreased from 71±6 to 30±9. A similar trend is observed for synthesis of oligo(L-Arg-co-L-Leu) (FIG. 9). That is, for α-chymotrypsin catalyzed co-oligomerizations of 1/1 L-Arg-Et.2HCl-to-L-Leu-Et.HCl, maximum %-yield and %-Arg incorporation in oligo(L-Arg-co-L-Leu) were obtained at pH 10 and 11, respectively. Comparison of maximum values of %-Lys and %-Arg values were similar (71±6 and 69±2, respectively). Furthermore, maximum incorporation of Lys and Arg in copolypeptides with Leu units is 35±1 and 34, respectively. Similarities of %-yield and %-compositions of oligopeptides formed from α-chymotrypsin catalyzed L-Lys-Et.2HCl/L-Leu-Et.HCl and L-Arg-Et.2HCl/L-Leu-Et.HCl oligomerization indicate that the relative reactivity ratios of the two monomer pairs are close in value.

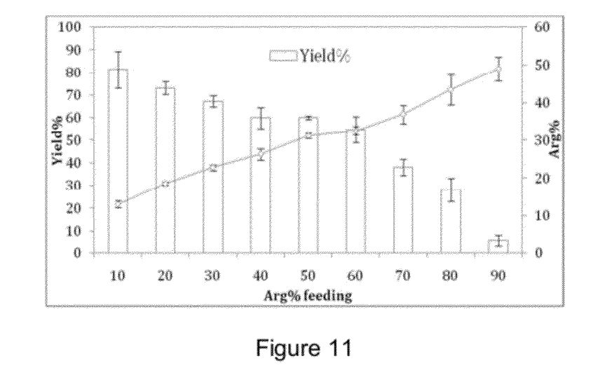

FIG. 10 displays results of %-yield and %-Lys incorporation in L-Lys-co-L-Leu oligopeptides as a function of the comonomer feed ratio. Oligomerizations were catalyzed by α-chymotrypsin with total monomer concentration 0.5M at pH 10.0. The product formed precipitates from the reaction during oligomer synthesis. As %-molar content of L-Lys-Et increases from 10 to 90%, the %-yield of co-oligopeptide decreases from 83±3 to 4% while %-Lys incorporation increases from 14±1 to 45%. Indeed, a similar trend was found for α-chymotrypsin-catalyzed L-Arg-Et.2HCl/L-Leu-Et.HCl oligomerizations as a function of monomer feed ratio at pH 10 (FIG. 11). That is, as %-molar content of L-Arg-Et increases from 10 to 90%, the %-yield of co-oligopeptide decreases from 81±8 to 6±2 while %-Arg incorporation increases from 13±1 to 49±3.

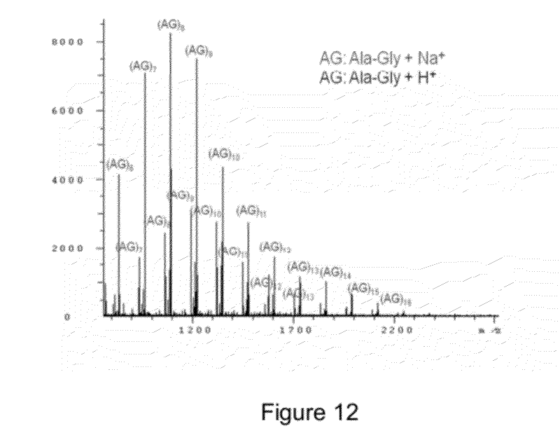

A chemoenzymatic route was devised by which a dipeptide was first synthesized by chemical methods after which it was oligomerized by protease-catalysis to form regularly alternating co-oligopeptides. As an example, the MALDI-TOF spectrum of alternating oligopeptide (Ala-Gly)ₙ, synthesized from Ala-Gly ethyl ester by α-chymotrypsin catalysis is displayed in FIG. 12. The oligomerization was conducted at pH 7, for 2 h from 0.25M Ala-Gly ethyl ester. Study of FIG. 12 shows two series of peaks separated by 128.1 corresponding to the mass of Ala-Gly. These two peaks originate due to formation of peptide H⁺ (denoted in blue) and Na⁺ (denoted in red) ions. A distribution of alternating oligopeptides were formed with DP values ranging from 12 to 32 units (i.e. having structures (Ala-Gly)₆ to (Ala-Gly)₁₆).chain lengths. The results of this experiment demonstrate that the predominant mechanism of chain formation is Ala-Gly ethyl ester oligomerization. In other words, MALDI-TOF spectra show no evidence that potential competitive pathways are occurring that would lead to non-alternating structures. Examples of such pathways include transamidation between oligopeptide chains and hydrolysis of monomer (L-Ala-Gly-Et) to Ala and Gly-Et that could subsequently be oligomerized.

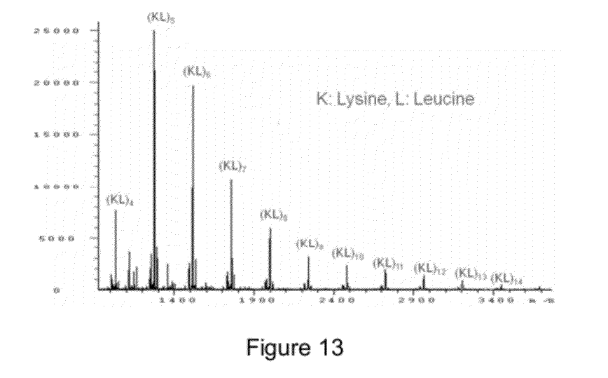

A chemoenzymatic route was devised by which a dipeptide was first synthesized by chemical methods after which it was oligomerized by protease-catalysis to form regularly alternating co-oligopeptides. As an example, the MALDI-TOF spectrum of alternating oligopeptide (Lys-Leu)ₙ synthesized from Lys-Leu-Et by α-chymotrypsin catalysis is displayed in FIG. 13. The oligomerization was conducted at pH 9 with 0.25M substrate concentration.

The reaction was rapid such that, 1 min after addition of α-chymotrypsin, the reaction mixture formed a gel. Study of FIG. 13 shows peaks separated by 241.2 corresponding to the mass of Lys-Leu. These peaks originate due to formation of Na⁺ ions. A distribution of alternating oligopeptides were formed with DP values ranging from 8 to 28 units (i.e. having structures (Lys-Leu)₄ to (Lys-Leu)₁₄). The results of this experiment demonstrate that the predominant mechanism of chain formation is Lys-Leu-Et oligomerization. In other words, MALDI-TOF spectra show no evidence that potential competitive pathways are occurring that would lead to non-alternating structures. Examples of such pathways include transamidation between oligopeptide chains and hydrolysis of monomer (L-Lys-Leu-Et) to Lys and Leu-Et that could subsequently be oligomerized.

L-Lys-co-L-Leu oligomerizations catalyzed by α-chymotrypsin were conducted at pH 10 with total monomer concentration 0.5M using a 1:1 molar feed ratio of L-Lys-Et.2HCl and L-Leu-Et.HCl. The product was fractionated to obtain a part of the product with enhanced activity. This was achieved by adding 20 mL deionized water to the reaction mixture. The solution was then titrated to pH 1 by adding 6 M HCl. After centrifuge at 8000 rpm for 8 min, the insoluble precipitate was removed. By adjusting the supernatent to pH 11 at 25° C., the clear solution turned turbid. This cloudy solution was centrifuged at 8000 rpm for 8 min to give an insoluble fraction that was collected, lyophilized, and used for antimicrobial assays. Details of the procedure for antimicrobial activity assay measurements are described above. In summary, antimicrobial assays were performed using 96 well micro-plates with 10 human pathogens including 9 bacteria and 1 yeast culture. Bacteria used were 11 human pathogens including 10 bacteria and 1 yeast culture. Bacteria used were Proteus milrabillis, Staphylococcus aureus, Pseudomonas aeruginosa, Bacillus subtilis, Escherichia coli, Klebsilla pneumonia, Nesseria gonoria, Morexella sp., Proteus vulgaris, Streptococcus neumonia. The yeast used was Candida tropicalis. Peptide stock solutions (100 μL) were placed in the first row of wells in the micro-plate and serially diluted using BHIB. An 80 μL aliquot of fresh culture medium was transferred into the wells having 100 μL of diluted peptide solution to bring the total volume to 180 μL. Microbial cultures were grown in BHIB for 18 h at 37° C. and diluted using the same fresh culture medium to obtain an OD of 0.5 at 540 nm. A 20 μL aliquot of diluted bacterial and yeast cultures (with OD of ˜0.5) were added in to the above wells with serially diluted peptide solution and incubated for 24 h at 37° C. In order to determine the growth of cultures without peptide, control cultures were conducted by replacing 100 μL peptide solutions with 100 μL culture broth (100 μL). Also, the antibiotic streptomycin was used as a standard to compare the antimicrobial activity of peptides. Antimicrobial activity was estimated by measuring the OD of the above micro-plates with microbial culture and peptide solution at 540 nm. OD measured immediately after inoculation of the bacterial culture was taken as 0 h. Subsequently, OD measurements were recorded at 12 and 24 h after incubation. OD obtained in control cultures without peptide was compared with OD observed with peptides, the difference in the OD was converted into percentage (%) and the antimicrobial activity was expressed in % of growth inhibition. MIC was determined from above results and is defined as the peptide concentration that inhibits microbial growth by more than 95% (Table 4).

Previous work with model oligopeptides consisting of Leu (L) and Lys (K) units provides information on sequences to obtain antimicrobial LK products. The amphipathic peptide LKLLKKLLKKLKKLLKKL, synthesized by traditional chemical methods, was designed to readily adopt an α-helical conformation upon interacting with lipid membranes resulting in antimicrobial activity. This peptide shows better antimicrobial activity on S. aureus, E. coli, and P. aeruginosa than the diblock peptide KKKKKKKKKLLLLLLLLL or the alternating sequence peptide KLKLKLKLKLKLKLKLKL.⁶² Other linear model peptides (12 amino acids long) with varying Lys:Leu (or tryptophan) contents (i.e., K₃L₈W, K₅L₆W, and K₇L₄W) also confer different levels of antibacterial activity and cell selectivity.⁶³ In a minimalist approach to modeling lytic toxins, amphipathic peptides of LᵢKⱼ with i=2j and whose length varies from 5 to 22 residues were studied for their ability to induce hemolysis and lipid vesicle leakage.⁶⁴ Antimicrobial peptides with repeating sequences (KLAKKLA)ₐ, (KLAKLAK)ₐ, (KALKALK)₃, (KLGKKLG)_{b}, and (KAAKKAA)_{b} (where a=1, 2, 3 and b=2, 3) were designed to have amphipathic structures which adopt helical conformations. Their antibacterial activity was tested against E. coli, P. aeruginosa, and S. aureus.⁶⁵ Generally, the antimicrobial activity of the above peptides with uniform sequence and chain length, prepared by traditional chemical methods, is higher than that determined for amphipathic peptides prepared by protease catalysis (see Table 4).

Through further iteration of the methods described herein it is expected that amphipathic peptides from protease-catalysis will be developed with activities closely matching or even exceeding those of their chemical counterparts. Even though L-K antimicrobial peptides produced by protease catalysis by the methods disclosed in this invention have generally lower antimicrobial activity than the above peptides produced traditional chemical preparative methods (e.g. solid or liquid state synthetic methods), uniform sequence and chain length peptides produced by traditional chemical methods require tedious multiple step processes that are time consuming, chemically intensive and costly. The high cost of uniform sequence and chain length peptides extracted from natural sources or prepared by traditional chemical approaches is well known by one skilled in the art. For example, Melittin, a natural antimicrobial peptide from bee venom, is available from Sigma Aldrich for $895 per milligram (see Jan. 24, 2011, www.sigmaaldrich.com). In contrast, peptide mixtures prepared by protease catalysis will be much lower in cost than uniform sequence and chain length peptides while delivering useful antimicrobial activity. Since they are produced by protease catalysis using simple building blocks (amino acid alkyl esters) under mild conditions, the costs of production of such products will be much lower.

Table 4 lists the antimicrobial activity, expressed as %-growth inhibition, by a L-Lys-co-L-Leu oligopeptide obtained by α-chymotrypsin catalyzed oligomerization of a 1:1 molar feed ratio of L-Lys-Et.2HCl and L-Leu-Et.HCl. This oligopeptide exhibited 20 to 47% growth inhibition against all 11 (10 bacterial and 1 yeast) pathogens used. This demonstrates that L-Lys-co-L-Leu peptide mixtures synthesized by protease-catalysis based on the structural motif of Lys-Leu model peptides described above have broad spectrum activity against many human pathogens. The maximum growth inhibitory activity of 47% was observed against the pathogen E. coli with a peptide concentration of 2 mg/mL. Those skilled in the art can use the developed methods herein to further improve the antimicrobial activity of amphipathic peptides synthesized by protease-catalysis.

### Illustrative Processes:

The present invention has a broad range of uses and encompasses the novel aspect that unique peptide mixtures can be synthesized by protease-catalyzed oligomerization of natural and non-natural amino acid alkyl ester monomers and specific combinations of these monomers as dimers, trimers and higher oligomers, in order to incorporate structural motifs that mimic those found in natural peptides or that were the result of studies of uniform sequence and length peptides prepared by other methods that include but are not limited to solid state peptide synthesis, isolation of peptides from natural sources, and production of peptides by recombinant DNA methods. Furthermore, this invention discloses that said peptide mixtures produced by protease catalysis with incorporated structural motifs can replicate to various extents the properties found in corresponding peptides with uniform sequences and lengths prepared by the above discussed methods.

One embodiment of the present invention is a process for preparing oligopeptide mixtures that mimic structural motifs and, therefore, physical/biological properties of peptides having uniform sequence and length that comprises the following steps.
Admixing one or more natural and non-natural amino acid alkyl ester monomer, dimer, trimer and higher oligomers with one or more proteases in a reaction medium;
Heating the mixture to between about 5° C. to about 90° C. for between 5 minutes and 24 hours; and
Recovering the synthesized oligopeptide from the reaction mixture.

Further downstream processing steps may include removal of alkyl ester groups and fractionation of oligopeptides.

An embodiment of the process employs an amino acid alkyl ester having the following general formula (1):

H₂N—CH(R)—(CR′H)ₙ—COOX (1)

in which R represents an amino acid side chain, R′ represents a different amino acid side chain present in β-amino acids, and X is an alkyl ester preferably consisting of an alkyl group selected from those containing from one to six carbon atoms but may consist of up to 20 carbon atoms. The alkyl ester may be straight or branched chain and include methyl, ethyl, propyl, isopropyl, butyl, hexyl and the like. In a preferred embodiment, the alkyl ester is selected from the group consisting of methyl, ethyl or propyl groups. Activated esters can also be used in place of alkyl esters, and examples of activated esters include guanadinophenyl, p-nitrophenyl, 1,1,1,3,3,3,-hexafluoroisopropyl, 2,2,2-triifluoroethyl, 2-chloro ethyl ester, carbamoyl methyl ester, benzyl esters, and anilides. Another embodiment of the process is where in the general formula (1) n=0, the stereochemistry is L, and R is selected from one or more of the natural amino acid side chains.

Another embodiment of the present invention is a process that employs an amino acid alkyl ester having the general formula (2):

H₂NCH₂CH(R)—COOX (2)

wherein the β-amino acids and other non-natural amino acid structures are those known to those of ordinary skill in the art as useful substrates for protease-catalyzed oligopeptide synthesis or protease-catalyzed coupling of preformed segments of oligo(amino acids). Illustrative β-amino acids are those that consist of the general structure (2) and have R groups selected from the above Chart 1. Examples of β-amino acids include: β-alanine, l-β-homotyrosine, l-β-homoleucine, l-β-homoisoleucine and l-β-homotryptophan. Examples of other non-natural amino acid esters include: carnitine [3-Hydroxy-4-trimethylammonio-butanoate], ornithine [(+)-(S)-2,5-diamino valeric acid], citruline [2-Amino-5-(carbamoylamino)pentanoic acid], 4-aminobutanoic acid and L-Dopamine, and other non-natural amino acids.

The most preferable alkyl ester group X in general formula (1) is ethyl.

The reaction medium used can consist of a phosphate, acetate, borate, carbonate, HEPES, or sulphate buffers with concentrations that can vary widely but generally are between 0.1 M to 1.5M. Or, instead of buffer salts, some amines such as triethyl amine can be used to maintain reaction medium pH. A water-miscible cosolvent selected from the group consisting of formamides, alcohols (primary, secondary, and tertiary), dimethyl sulfoxide, tetrahydrofuran, acetone, acetonitrile, 1,2-ethylene glycol, 1,3-propylene glycol, or 1,4-butanediol can be added in concentrations from 0 to 50%-v/v.

In another embodiment of the present invention, the enzyme or enzyme mixture is selected from a member of a hydrolytic enzyme family that is further comprised of proteases, lipases, esterases and cutinases. The enzyme can be selected from members of the protease family, and wherein:

(i) suitable proteases for use in this invention include papain, bromelain, α-chymotrypsin, trypsin, Multifect P-3000 (Genencor), Purafect prime L (Genencor), alkaline protease (Genencor), metalloprotease (thermolysin), protease from subtilisin (family), pronase1, glutaminase, carboxypeptidase Y, clostrapin, protease from aspergillus oryzae species, pepsin, cathepsin, ficin, alcalase, carboxypeptidase, calpains, actinidin, chymosin, carbonic anhydrase, nonribosomal peptide synthetase, thrombin, cardosins A or B or pronase;

(ii) a reaction can be catalyzed by one or a mixture of 2 or more proteases;

(iii) variants of these enzymes, generated by standard protein engineering methods such as error-prone PCR and gene shuffling, well known to those of ordinary skill in the art, can be used to further improve a proteases activity and selectivity for use in the current invention; and

(iv) suitable enzymes may be identified by other methods known by those skilled in the art, can be identified via searches of gene data banks, can subsequently be synthesized by preparation of the gene, cloning of the gene into a suitable host, and production of the enzyme by fermentation, and may be identified by DNA mining from various environments such as in soil.

The enzymes can be added to the reaction media as enzyme powders, in solution, or immobilized on a support.

The reaction can be terminated in various manners. For example, the reaction can be terminated by filtration of the immobilized enzyme. The reaction also can be terminated by separation of the precipitated end-functionalized oligopeptide product by filtration or centrifugation from the enzyme remaining in the reaction medium. The reaction also can be terminated by using a membrane with a suitable pore size that separates a soluble end-functionalized oligopeptide product from the soluble enzyme. The reaction can be terminated by selective precipitation of either the soluble enzyme or the soluble oligopeptide product.

The reaction time preferably is between 5 minutes and 24 hours. More preferably, the reaction time is between 10 minutes and 8 hours. Even more preferably, the reaction time is between 30 minutes and 3 hours.

The reaction temperature is between 5° C. and 90° C. More preferably, the reaction temperature is between 25° C. and 60° C. Even more preferably, the reaction temperature is between 30° C. and 40° C.

In another embodiment of the invention, the reaction is performed by passing reactants through a column wherein the stationary phase consists of the immobilized enzyme.

The oligopeptides can consist of a mixture of oligomers. For example, the oligopeptides can consist of a mixture of oligomers where the average chain length, determined by measuring the number average molecular weight, ranges from 2 to 100 units. Preferably, the oligopeptides consist of a mixture of oligomers where the average chain length, determined by measuring the number average molecular weight, ranges from 5 to 50 units. More preferably, the oligopeptides consist of a mixture of oligomers where the average chain length, determined by measuring the number average molecular weight, ranges from 10 to 20 units.

Additionally, the oligopeptides can consist of a mixture of oligomers having a certain polydispersity. For example, the oligopeptides consist of a mixture of oligomers with a polydispersity, determined by dividing the weight average molecular weight by the number average molecular weight, of 50. Preferably, the oligopeptides consist of a mixture of oligomers with a polydispersity, determined by dividing the weight average molecular weight by the number average molecular weight, that is <25. More preferably, the oligopeptides consist of a mixture of oligomers with a polydispersity, determined by dividing the weight average molecular weight by the number average molecular weight, that is <5. Even more preferably, the end-capped oligopeptides consist of a mixture of oligomers with a polydispersity, determined by dividing the weight average molecular weight by the number average molecular weight, that is <1.5.

In another embodiment of the invention, the oligomers are useful for metal binding and are prepared from co-oligomerization of amino acid 1 (AA1) and amino acid 2 (AA2), wherein AA1 is L-Et₂-glutamic acid and AA2 is the ethyl ester derivative selected from the group consisting of L-histidine, L-cysteine, L-lysine, L-asparagine, or L-aspartic acid. A preferred AA2 is L-cysteine ethyl ester. The oligopeptide preferably comprises from 30 to 70 mol % of L-γ-Et-glutamic acid units and 70 to 30 mol % of L-cysteine units. The oligopeptide more preferably comprises from 40 to 60 mol % of L-γ-Et-glutamic acid units and 60 to 40 mol % of L-cysteine units. The oligopeptide even more preferably comprises from 45 to 55 mol % of L-γ-Et-glutamic acid units and 55 to 45 mol % of L-cysteine units.

In yet another embodiment of the invention, the oligomers are useful for antimicrobial activity and are prepared from co-oligomerization of amino acid 1 (AA1) and amino acid 2 (AA2), wherein AA1 is L-lysine ethyl ester or L-arginine ethyl ester and AA2 is the ethyl ester derivative selected from the group consisting of L-alanine, L-valine, L-leucine, L-isoleucine, or L-phenylalanine. The oligopeptide preferably comprises from 20 to 50 mol % of AA1 units and 80 to 50 mol % of AA2 units. The oligopeptide more preferably comprises from 30 to 50 mol % of AA1 units and 70 to 30 mol % of AA2 units. The oligopeptide even more preferably comprises from 40 to 50 mol % of AA1 units and 60 to 50 mol % of AA2 units.

The process also can comprise fractionation of synthesized co-oligomer mixtures to obtain a product mixture with enhanced physical or biological activity. The fractionation can be achieved by centrifugation filters with predefined molecular cut-off values to obtain desired product fractions. The fractionation also can be achieved by differential solubility using common organic solvents selected from the group consisting of methanol, ethanol, 1-propanol, isopropanol, acentonitrile, 1,4-dioxane, chloroform, THF, DMSO and DMF, and combinations thereof. The fractionation also can be achieved by shifts in solution pH with or without variation in the ionicity or nature of the cationic species. The fractionation also can be achieved by exploiting different molecular weights or hydrodynamic volumes of constituents in the product mixture and such fractionation is achieved by size exclusion chromatograph (SEC).

In an embodiment of the invention, the natural and non-natural amino acid alkyl esters are selected from the group of structural motifs of metal binding, adhesion, self-assembly, antimicrobial activity, protein inhibition, ingredients in cosmetic formulations, and peptide therapeutics.

Another embodiment of the process of the present invention can further comprise adding a molecule to the mixture of reactants that end-functionalizes the N-terminus, C-terminus or both ends of the peptide, wherein the general formula for the synthesized oligomer is C-peptide-B wherein B is a group at the carboxyl terminus and C is a group at the N-terminus. A preferred end-functionalization agent comprises an activated ester with the structure:

Y′Y—[H]ₐN—CH(R)—(CR′H)ₙ—COOX (3)

wherein
X is a straight or branched chain alkyl ester consisting of an alkyl group selected from those containing from 1 to 20 carbon atoms, or is an activated ester,
Y and/or Y′ is selected from H, methyl, ethyl, CH₂═CH—CO—, CH₂═C(CH₃)—CO—, HOOC—CH═CH—CO— (cis or trans), functional groups used for photolytic crosslinking, cinnamoyl (Ph-CH═CH—CO—) group, groups that are crosslinkable via redox catalysts, and HO-Ph-(CH₂)—CO— where the hydroxyl group is at the para-position.

In certain embodiments, Y and/or Y′ is selected from structures that are used in bioconjugate chemistry. Preferably, Y and/or Y′ is selected from the group consisting of alkyne functionalized molecules, azide functionalized molecules, and terminal alkene functionalized molecules. For example, Y and/or Y′ can be selected from the group consisting of 4-alkyne-pentanoate (HC≡C—CH₂—CH₂—CO—), N₃—CH₂—CH₂—CH₂—CH₂—CH₂—CO—), and 2-propen-1-amine. For another example, n=0, Y is H, Y′ is CH₂═CH—CO— or CH₂═C(CH₃)—CO—, and R is selected from the group consisting of glycine, alanine, valine, leucine, isoleucine, serine, threonine, cysteine, methionine, proline, phenylalanine, tyrosine, tryptophan, histidine, lysine, arginine, aspartic acid, asparagine, glutamic acid, glutamine, and combinations thereof.

Another embodiment of the invention is a process for preparing oligopeptides end-functionalized at the N-terminus, C-terminus or at both ends and that has the general formula C-peptide-B, wherein B is a group at the carboxyl terminus, C is a group at the N-terminus, comprising the steps of:
a) admixing at least one natural and at least one non-natural amino acid alkyl ester monomer, dimer, trimer, or higher oligomers with at least one enzyme in a reaction medium;
b) initiating a reaction by heating the mixture to between about 5° C. to about 90° C. for between 5 minutes and 24 hours; and
c) recovering the oligopeptide.

This embodiment can further comprise performing a modification of the N-terminal amino group by conventional coupling methods using conventional chemical methods. For example, the N-terminal group of oligopeptides can be modified by N-acylation chemistry using conventional chemical methods.

One example of this embodiment is when the N-acylated amino acids formed has the structure:

R(C═O)NH-peptide-COOX (4)

wherein R(C═O) is derived from a natural fatty acid selected from the group consisting of lauric acid (dodecanoic acid), myristic acid (tetradecanoic acid), palmitic acid (hexadecanoic acid), palmitoleic acid (9-hexadecenoic acid), stearic acid (octadecanoic acid), oleic acid (9-octadecenoic acid), ricinoleic acid (12-hydroxy-9-octadecenoic acid), linoleic acid (9,12-octadecadienoic acid), α-linolenic acid (9,12,15-octadecatrienoic acid), γ-linolenic acid (6,9,12-octadecatrienoic acid), behenic acid (docosanoic acid), and erucic acid (13-docosenoic acid).

The fatty acid can be first modified by hydrogenation, epoxidation, or hydroxylation prior to reaction with NH₂ terminal groups of oligopeptides. In an exemplary embodiment, R is selected from the group consisting of —CH₂═CH—CO—, CH₂═C(CH₃)—CO—, and HOOC—CH═CH—CO— (cis or trans).

Another example of this embodiment is when the end-functionalization agent comprises an amine having the structure:

H₂N—CH(R)—(CR′H)ₙ—COHN—Z (3)

wherein n=0,
wherein R is selected from the group consisting of lauric acid (dodecanoic acid), myristic acid (tetradecanoic acid), palmitic acid (hexadecanoic acid), palmitoleic acid (9-hexadecenoic acid), stearic acid (octadecanoic acid), oleic acid (9-octadecenoic acid), ricinoleic acid (12-hydroxy-9-octadecenoic acid), linoleic acid (9,12-octadecadienoic acid), α-linolenic acid (9,12,15-octadecatrienoic acid), γ-linolenic acid (6,9,12-octadecatrienoic acid), behenic acid (docosanoic acid), and erucic acid (13-docosenoic acid), and
wherein Z is selected from structures that are used in bioconjugate chemistry.

Illustrative examples of Z are selected from the group consisting of maleimide functionalized molecules for thiol-maleimide chemistry, azide functionalized molecules for azide alkyne chemistry, alkyne functionalized molecules for azide alkyne chemistry. For example, Z can be selected from the group consisting of —CH₂—CH₂-maleimide, —CH₂—CH₂—N₃), and —CH₂≡CH.

Another embodiment of the present invention comprises a specific combination of two amino acids selected from the natural and non-natural amino acids based on synthesizing oligopeptide mixtures by protease catalysis for a desired structural motif, the dimer of the two amino acids selected is synthesized by chemical or enzymatic methods, and then the dimer alkyl ester is used as monomer to prepare alternating co-oligopeptides.

Some additional examples of oligomerization by protease catalysis contemplated by the present invention include the general process of:
a) admixing at least one natural and at least one non-natural amino acid alkyl ester monomer, dimer, trimer, or higher oligomers with at least one enzyme in a reaction medium;
b) initiating a reaction by heating the mixture to between about 5° C. to about 90° C. for between 5 minutes and 24 hours; and
c) recovering the oligopeptide.
   wherein dimers of L-Ala and L-Gly are prepared and then the corresponding dipeptide ethyl ester can be oligomerized by protease catalysis to form alternating peptides of the composition -(-L-Ala-L-Gly-)ₙ-. L-Ala-L-Gly ethyl ester can be prepared by esterification of L-Ala-L-Gly in ethanol with thionyl chloride. Dimers of L-Lys and L-Leu can be prepared and then the corresponding dipeptide ethyl ester is oligomerized by protease catalysis to form alternating peptides of the composition -(-L-Lys-L-Leu-)ₙ-. Boc-L-Lys(Boc)-L-Leu-ethyl ester can be prepared by coupling Boc-Lys(Boc)-OSu with leucine ethyl ester at room temperature catalyzed by triethylamine. Boc-L-Lys(Boc)-L-Leu-ethyl ester can be deprotected by using dichloromethane (DCM)/trifluoroacetic acid(TFA) as co-solvents in a 3:1 v/v ratio.

Alternatively, dimers of L-Lys and L-Phe can be prepared and then the corresponding dipeptide ethyl ester can be oligomerized by protease catalysis to form alternating peptides of composition -(-L-Lys-L-Phe-)ₙ-. Boc-L-Lys(Boc)-L-Phe-ethyl ester can be prepared by coupling Boc-Lys(Boc)-OSu with phenylalanine ethyl ester at room temperature catalyzed by triethylamine. Boc-L-Lys(Boc)-L-Phe-ethyl ester can be deprotected by using dichloromethane (DCM)/trifluoroacetic acid(TFA) as co-solvents in a 3:1 v/v ratio.

In other embodiments of the invention, the end-functionalization agent comprises an amine having the structure:

H₂N—CH(R)—(CR′H)ₙ—COHN—Z (3)

and an activated ester having the structure:

Y′Y—[H]ₐN—CH(R)—(CR′H)ₙ—COOX

wherein n=0,
wherein R is selected from the group consisting of glycine, alanine, valine, leucine, isoleucine, serine, threonine, cysteine, methionine, proline, phenylalanine, tyrosine, tryptophan, histidine, lysine, arginine, aspartic acid, asparagine, glutamic acid, glutamine, and combinations thereof, and
wherein Z is selected from the group consisting of an azide functionalized moiety,
wherein Y is H, and
wherein Y′ is an azide functionalized moiety or an alkyne functionalized moiety.

Z and Y can be selected to be used in combination to prepare oligopeptides that are functionalized at both the amino- and carboxyl-termini. For example, Z can be —CH₂—CH₂—N₃), Y can be H, and Y′ can be N₃—CH₂—CH₂—CH₂—, CH₂—CH₂≡CH, or HC≡C—CH₂—CH₂—CO—.

Another embodiment of the invention includes the general process wherein an oligopeptide mixture, synthesized by protease-catalysis, can be functionalized at either the N- or C-terminus with groups useful in bioconjugate chemistry, and end-functionalized groups can be used to conjugate the peptides to substances. For example, the general process can further comprise:

functionalizing an oligopeptide mixture, synthesized by protease-catalysis, at either the N- or C-terminus with groups useful in bioconjugate chemistry, and
coupling end-functionalized peptides to chain segments of natural, synthetic or hybrid polymer chains,
thereby resulting in coupled products consisting of: peptide mixtures; peptides of uniform chain length and sequence synthesized by chemical methods, isolated from nature, or produced by recombinant DNA methods; synthetic polymers such as heterobifunctional polyethylene glycol; and chain segments of DNA; and oligo- or polysaccharides such as those belonging to members of the glycosaminoglycan family, chitosan, pectin and amylose.

Another embodiment of the invention includes the general process further comprising:

functionalizing an oligopeptide mixture, synthesized by protease-catalysis, at both the N- and C-terminus with groups useful for bioconjugate chemistry, and
copolymerizing end-functionalized peptides with at least one chain segments that has suitable functional groups at both chain termini selected from the group consisting of: peptides of uniform chain length and sequence synthesized by chemical methods, isolated from nature, or produced by recombinant DNA methods; synthetic polymers such as end-functionalized polyethylene glycol, oligo- or polylactic acid, oligo- or polythiopene; chain segments of DNA; and oligo- or polysaccharides such as those belonging to members of the glycosaminoglycan family, chitosan, pectin and amylose.

The above detailed description of the preferred embodiments, examples, and the appended figures are for illustrative purposes only and are not intended to limit the scope and spirit of the invention, and its equivalents, as defined by the appended claims. One skilled in the art will recognize that many variations can be made to the invention disclosed in this specification without departing from the scope and spirit of the invention.

### REFERENCES

1. DeSilva, T. M., et al., J. Biopolymers 2002, 64, 189-197.
2. Summers, A. O., Annual Review of Microbiology 1986, 40, 607-634.
3. Cheng, Y., et al., Journal of Inorganic Biochemistry 2005, 99, 1952-1962.
4. Rauser, W. E., Annu. Rev. Biochem. 1990, 59, 61-86.
5. Dannenberg, A. M., et al., J. Biol. Chem. 1955, 215, 1955.
6. Raskin, I., Proceedings of the National Academy of Sciences of the United States of America 1996, 93, 3164-3166.
7. Buchsbaum, J. C., et al., Inorg. Chim. Acta 2000, 297, 217-219.
8. Åuczkowski, M., et al., Inorganic Chemistry 2008, 47, 10875-10888.
9. Hof, W. v. t., et al., Biol. Chem. 2001, 382, 597-619.
10. Tossi, A., et al., Peptide Science 2000, 55, 4-30.
11. Marsh, J., et al., Ciba Foundation Symposium 186—Antimicrobial Peptides, Wiley Online publisher 1994, Vol. 186.
12. Yang, L., et al., Biophys. J. 2000, 79, 2002-2009.
13. Cummings, J. E., Interactions of the antimicrobial peptide cryptdin-4 with phospholipid membranes, Princeton University, New Jersey, 2007.
14. Zasloff, M., Nature 2002, 415, 389-395.
15. O'Brien-Simpson, N. M., et al., Journal of the American Chemical Society 1997, 119, 1183-1188.
16. Gill, I., et al., Enzyme Microb. Technol. 1996, 18, 162-183.
17. Maskarinec, S. A., et al., Current Opinion in Biotechnology 2005, 16, 422-426.
18. Gill, I., et al., Enzyme Microb. Technol. 1996, 18, 162-183.
19. Deming, T. J., Prog. Polym. Sci. 2007, 32, 858-875.
20. Strøm, M. B., et al., Journal of Medicinal Chemistry 2003, 46, 1567-1570.
21. Mohan, K. V. K., et al., Transfusion 2010, 50, 166-173.
22. Kisiday, J., et al., PNAS 2002, 99, 9996-10001.
23. Fairman, R., et al., Curr. Opin. in Struc. Biol. 2005, 15, 453-463.
24. Guzmán, F., et al., Electronic Journal of Biotechnology 2007, 10, 279-314.
25. Bordusa, F., Chem. Rev. 2002, 102, 4817-4868.
26. Yeaman, M. R., et al., Pharmacological Reviews 2003, 55, 27-55.
27. Hancock, R. E. W., et al., Antimicrob. Agents Chemother. 1999, 43, 1317-1323.
28. Tamerler, C., et al., Materials Research Society 2003, 773.
29. Shi, Y., et al., Biophys. J. 1993, 64, 749-753.
30. Wilson, D., et al., Biophys. J. 2000, 78, 2690-2701.
31. Kar, K., et al., Protein Sci. 2008, 17, 1086-1095.
32. Tamamis, P., et al., The Journal of Physical Chemistry B 2009, 113, 15639-15647.
33. Ruoslahti, E., et al., Science 1987, 238, 491-497.
34. Lupo, M. P., et al., Dermatologic Therapy 2007, 20, 343-349.
35. Katayama, K., et al., J. Biol. Chem. 1993, 268, 9941-9944.
36. Jun, H. W., et al., J. Biomater. Sci. Polymer Edn. 2008, 19, 665-676.
37. Zhao, X., et al., ChemInform 41, no-no.
38. Fraser, R. D. B., et al., Conformation of Fibrous Proteins and Related Synthetic Polypeptides (Molecular Biology), Academic Press New York, 1974, p 630.
39. Ayres, L., et al., Biomacromolecules 2005, 6, 825-831.
40. Smeenk, J. M., et al., Angew. Chem. Int. Ed. 2005, 44, 1968-1971.
41. Meister, A., Proteinase catalyzed synthesis of peptide bonds, John Wiley & sons, New York, 1992, Vol. 53, p 239-306.
42. Ferjancic, A., et al., Appl. Microbiol. Biotechnol. 1990, 32, 651-657.
43. Ferjancic, A., et al., Biotechnol. Lett 1991, 13, 161-166.
44. Uyama, H., et al., Biomacromolecules 2002, 3, 318-323.
45. Matsumura, S., et al., Macromol. Rapid Commun. 1999, 20, 7-11.
46. Soeda, Y., et al., Biomacromolecules 2003, 4, 196-203.
47. Li, G., et al., Macromolecules 2008, 41, 7003-7012.
48. Li, G., et al., Macromolecules 2008, 41, 7003-7012.
49. Viswanathan, K., et al., Macromolecules 43, 5245-5255.
50. Li, G., et al., Macromolecules 2006, 39, 7915-7921.
51. Isidro-Llobet, A., et al., Chem. Rev. 2009, 109, 2455-2504.
52. Li, G., et al., Macromolecules 2006, 39, 7915-7921.
53. Riddles, P. W., et al., Analytical Biochemistry 1979, 94, 75-81.
54. Yang, H., et al., J. Am. Soc. Mass. Spectrom. 2009, 20, 2284-2293.
55. Li, G., et al., Macromolecules 2006, 39, 7915-7921.
56. Luczkowski, M., et al., Inorg. Chem. 2008, 47, 10875-10888.
57. Mehra, W. B., et al., J. Inorg. Biochem. 1997, 68, 201-210.
58. Kobayashi, R., et al., Biol. Trace Elem. Res. 2006, 114, 313-318.
59. Giedroc, X., et al., J. Biol. Inorg. Chem. 2000, 5, 93-101.
60. Yoshimura, E., et al., Chem. 2000, 38, 141-142.
61. Greenfield, N. J., et al., Biochemistry 1969, 8, 4108-4116.
62. Blondelle, S. E., et al., Biochemistry 1992, 31, 12688-12694.
63. Hong, J., et al., Biochemistry 1999, 38, 16963-16973.
64. Castano, S., et al., Biochimica et Biophysica Acta 1999, 1416, 161-175.
65. Javadpour, M. M., et al., J. Med. Chem. 1996, 39, 3107-3113.

CHART 1
Protein Amino Acids and Their Functional (R—) Groups
With Their 3-Letter & (1-Letter) Abbreviations

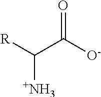

Neutral amino acids

| | |
|---|---|
| Glycine | H— |
| Gly (G) | |
| Alanine | H₃C— |
| Ala (A) | |
| Valine Val (V) | 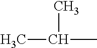 |
| Leucine Leu (L) | 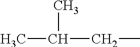 |
| Isoleucine Ile (I) | 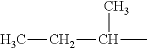 |
| Serine | HO—CH₂— |
| Ser (S) | |
| Threonine Thr (T) | 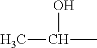 |

Sulfur amino acids

| | |
|---|---|
| Cysteine | HS—CH₂— |
| Cys (C) | |
| Methionine | H₃C—S—CH₂—CH₂— |
| Met (M) | |

Cyclic amino acids

| | |
|---|---|
| Proline Pro (P) | 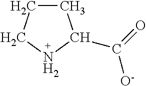 |

Aromatic amino acids

| | |
|---|---|
| Phenylalanine Phe (F) | 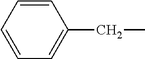 |
| Tyrosine Tyr (Y) | 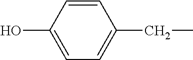 |
| Tryptophan Trp (W) | 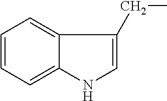 |
| Histidine His (H) | 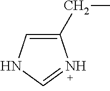 |

Basic amino acids

| | |
|---|---|
| Lysine Lys (K) | 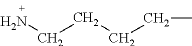 |
| Arginine Arg (R) | 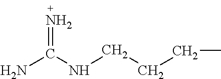 |

Acidic amino acids & their amides

| | |
|---|---|
| Aspartic acid Asp (D) | 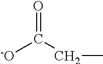 |
| Asparagine Asn (N) | 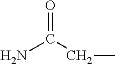 |
| Glutamic acid Glu (E) | 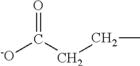 |
| Glutamine Glu (Q) | 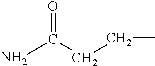 |

TABLE -I
Molecular characteristics of the inorganic-binding polypeptide sequences.

| | Charge at pH 7 [symbol] | PI [symbol] | M.W. [symbol] |
|---|---|---|---|
| Pd Binders | | | |
| 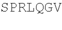 | +1 | 9.47 | 755.87 |
| TTLNPGT | 0 | 5.19 | 702.76 |
| VNSHPPL | 0 | 6.71 | 762.86 |
| TLPNHTF | 0 | 6.40 | 828.92 |
| Pt Binders | | | |
| 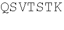 | +1 | 8.75 | 749.82 |
| PTSTGQA | 0 | 5.96 | 660.68 |
| 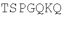 | +1 | 8.41 | 744.80 |
| 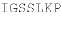 | +1 | 8.75 | 700.83 |
| Au Binders | | | |
| 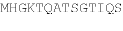 | +1 | 8.52 | 1446.60 |
| 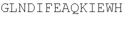 | −2 | 4.65 | 1699.88 |
| Ag Binders | | | |
| 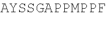 | 0 | 5.57 | 1221.39 |
| 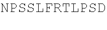 | 0 | 6.09 | 1395.53 |
| 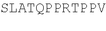 | +1 | 9.47 | 1263.46 |
| Silica Binders | | | |
| 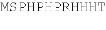 | +1 | 9.59 | 1470.63 |
| 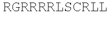 | +6 | 12.30 | 1541.89 |
| 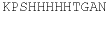 | +1 | 8.78 | 1359.43 |
| ZnO Binders | | | |
| 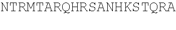 | +5 | 12.48 | 2351.59 |
| 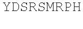 | +1 | 8.75 | 1148.26 |
| Cr₂O₃ Binders | | | |
| VVRPKAATN | +2 | 12.30 | 1134.35 |
| RIRHRLVGQ | +3 | 12.30 | |
| CoO Binders | | | |
| GRMQRRVAH | +3 | 12.30 | 1110.31 |
| LGKDRPHFH | +1 | 8.76 | 1106.25 |

In each peptide sequence, residues which have ionizable groups are boxed. [symbol]
The approximate net charges of each peptide at pH 7 were determined by the contribution of N-terminal amino group and C-terminal carboxyl group, one at each end of the chain, and also number of its ionizable R groups (ref. D. L. Nelson and M. M. Cox, Lehninger Principles of Biochemistry, 3rd ed. (Worth Publ., New York, 200). [symbol]
Isoelectric points and [symbol] Molecular masses of peptides are calculated using Compute pI/Mw tool (http://us.expasy.org/tools/pi_tool/html). [symbol]
indicates data missing or illegible when filed

TABLE 2
Primary sequences or structural motifs leading to peptide amphiphiles, how
they self-assemble, and applications of these

| Primary peptide sequences | Assembled nanostructures | Applications |
|---|---|---|
| Ac-AₘK-NH₂ | Nanorods, nanotubes, bilayers | Antimicrobial agents and stabilisation |
| | | of membrane proteins |
| Ac-VₘKₙ-NH₂ | Bilayers, nanotubes, nanovesicles | DNA immobilisation and stabilisation |
| | | of membrane proteins |
| Ac-GₘD [symbol] -OH | Nanotubes, nanovesicles | |
| Ac-A [symbol] D-OH | Nanoropes, nanotubes, | Nanofabrication |
| | nanovesicles | |
| Ac-V [symbol] D-OH | Nanotubes, nanovesicles | Stabilisation of membrane proteins |
| Ac-KA [symbol] -OH | Nanotubes, nanovesicles | Stabilisation of membrane proteins |
| Ac-GAVILRR-NH₂ | Nano [symbol] , spherical | Nanofabrication |
| | micelles | |
| Ac-I₃K-NH₂ | Nanotubes | Templates for silicification |
| Ac-L₃K-NH₂ | Nanovesicles | |
| KₘL [symbol] | Nanofibrils and gels | Foods, cosmetics and drug delivery |
| NH [symbol] -X [symbol] -H [symbol] R [symbol] -CONH₂ (X = L, W, F) | Nanoparticles | Gene delivery |
| Chol-H [symbol] R [symbol] | Core-shell nanomicelles | Gene delivery |
| Ac-(AF) [symbol] -H [symbol] K [symbol] -NH₂ | Core-shell nanomicelles | Gene and drug delivery |
| A₃₂H [symbol] K [symbol] | Core-shell nanoparticles | Gene delivery |
| Ac-A₂V₂L₃WE [symbol] -COOH | Spherical nanovesicles | Potential drug delivery vehicles |
| C₃₂-H₂₃-EVHHQKL | Nanofibrils | Potential use in bio-fabrication |
| C₃₀-H₂₄-C₄G₃S [symbol] RGD | Nanofibers | Mineralisation |
| C [symbol] -H₁₉-A₄G₂S [symbol] RGD | Nanofibers | pH responsive scaffolds |
| C₁₆-H₃₃-A [symbol] G₂EIKVAV-OH | Nanofibers | Nanofabrication |
| C₁₆-H₃₃-A₄K₄ | Nanofibers | Templates for silicification |
| C₁₀-H₃₄-C₄G₃S [symbol] RGD | Nanofibers | pH responsive scaffolds |
| C₁₀-H₃₁-W(A₄K)₃A | Spherical, worm-like micelles, | Scaffolds |
| | nanofibers | |
| C₁₆-H₃₃-O-VEVE | Nanobelts, nanoribbons | pH responsive materials |
| C₁₈-H₃₃-V₃A₃E₃ | Nanofibrillar gels | Cell culture scaffolds |
| C₁₈-H₃₃-V₃A₃K₃RGDS | Nanofibrillar gels | Cell culture scaffolds |
| Fmoc-RGD | Hydrogels | Cell culture scaffolds |
| C₁₆-H₃₃-KKK (X = A, G, L, K) | Nanofibrils and nanomicelles | Antimicrobial agents |
| C₁₆-H₃₃-LSQETFSDLWKLLPEN | Red-like micelles | Inhibit p53-MDM2 protein interaction |
| C₁₆-H₃₃-GTAGLIGQERGDS | Long nanofibers | Cell adhesion, migration and drug |
| | | delivery |
| Chol-G₃R₃ YGRKKERQRRR | Nanoparticles | Antimicrobial agent |
| RGDSKKLLA(K)-C₈H [symbol] -diacetylene- | Nanofibers | Nano patterning. Cell culture scaffold |
| C₁₂H₂₅ | | |
| C₁₆-H₃₃-KTTKS | | Anti-wrinkle |
| C₁₈-H₃₃-GQPR | | Anti-wrinkle |
| C₁₈-H₃₃-GHK | | Anti-wrinkle |
| Ac-AO(C₁₄H₂₃)AEAAEKAAKY- | | Stabilise membrane proteins |
| AAEAAEKAAKAO(C₁₆H₂ [symbol] )A-NH₂ | | |

[symbol]

indicates data missing or illegible when filed

TABLE 3
Examples of non-natural amino acids or stereochemical mixtures of natural amino acids.

| | | | |
|---|---|---|---|
| β-alanine | 4-aminobutanoic | D-Glutamine | D-Leucine |
| | acid | | |
| L-β-homotyrosine | L-Dopamine | D-Glutamic Acid | DL-Leucine |
| L-β-homoleucine | D-Alanine | DL-Glutamic Acid | D-tert-Leucine |
| L-β-homoisoleucine | D-2-Aminobutyric | D-Histidine | D-(−)-Norvaline |
| | Acid | | |
| L-β-homotryptophan | D-Arginine | L-Homoarginine | D-Pipecolic |
| | | Hydrochloride | Acid |
| carnitine [3-Hydroxy-4- | D-Asparagine | L- | D- |
| trimethylammonio- | | Homophenylalanine | Phenylalanine |
| butanoate] | | | |
| ornithine [(+)-(S)-2,5- | DL-Asparagine | L-Isoserine | D-Proline |
| diamino valeric acid] | (monohydrate) | | |
| citruline [2-Amino-5- | D-Aspartic Acid | DL-Isoleucine | D-Pyroglutamic |
| (carbamoylamino)pentanoic | | | Acid |
| acid] | | | |
| 4-Hydroxy-L-isoleucine | D-Tyrosine | DL-Valine | D-Threonine |
| D-Threonine | DL-Tyrosine | trans-4-Hydroxy-L- | DL-Threonine |
| | | proline | |
| DL-Threonine | D-Tryptophan | DL-Tryptophan | |

TABLE 4
Antimicrobial activity, expressed as %-growth inhibition,
by a L-Lys-co-L-Leu oligopeptide^{a} obtained by α-chymotrypsin
catalyzed oligomerization of a 1:1 molar feed ratio of L-Lys-Et•2HCl
and L-Leu-Et•HCl and subsequent fractionation.

| | Pathogens^{b} | | | | | | | | | | |
|---|---|---|---|---|---|---|---|---|---|---|---|
| | Pm | Sa | Pa | Bs | Kp | Ng | Msp | Pv | Sn | Ct | Ec |
| Inhibition^{c} | 23 | 20 | 21 | 20 | 22 | 24 | 24 | 26 | 27 | 22 | 47 |

^{a}peptide concentration 2 mg/mL.
^{b}Bacteria used were Proteus milrabillis (Pm), Staphylococcus aureus (Sa), Pseudomonas aeruginosa (Pa), Bacillus subtilis (Bs), Escherichia coli (Ec), Klebsilla pneumonia (Kp), Nesseria gonoria (Ng), Morexella sp. (Msp), Proteus vulgaris (Pv), Streptococcus neumonia (Sn). The yeast used was Candida tropicalis (Ct).
^{c}Percent growth inhibition is based on control samples with only culture and pathogen.

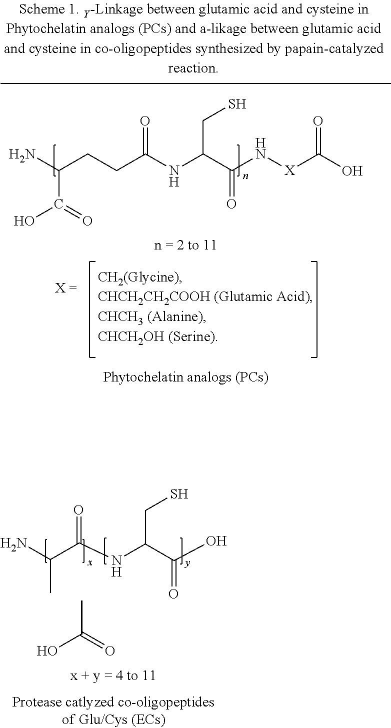

TABLE 5
Synthesis results of a papain-(16 units/mL) catalyzed reaction of
substrates, γ-L-(Et)₂-Glu•HCl and L-Et-Cys•HCl, in 5 mL, 0.9M
sodium phosphate buffer at pH 8.0, 40° C., 3 hr. Total substrate
concentration was 0.5M. Errors represent deviations from
the mean of at least two experiments, with the exception for E:C = 6:4.

| | | % cysteine | |
|---|---|---|---|
| Feed ratio [E:C] | Dp_{avg} | incorporation | % Yield^{a} |
| 10:0 | 8.2 | 0 | 70 ± 6 |
| 7:3 | 8.2 | 25 ± 2 | 44 ± 5 |
| 6:4 | 8.3 | 27 | 39 |
| 5:5 | 8 | 37 ± 3 | 40 ± 4 |
| 4:6 | 9.2 | 47 ± 4 | 34 ± 6 |
| 3:7 | 8.4 | 55 ± 5 | 22 ± 4 |
| 0:10 | 7.3 | NA | 25 ± 7 |

^{a}Evaluated with respect to total amount of monomer.

TABLE 6
Binding properties of metal complexes. Equilibrium dissociation
K_{d} and the product of the molar absorptivity change
(Δε) and the path length b are listed.

| Metal | | |
|---|---|---|
| complexes | K_{d} (mol L⁻¹) | bΔε (L mol⁻¹) |
| Cd(II) | 3.37E−04 | 1331 |
| Zn(II) | 3.38E−05 | 10300 |
| Ni(II) | 2.92E−04 | 607.7 |
| Co(II) | 7.60E−0.3 | 431.4 |

What is claimed is:

1. A process for preparing an oligopeptide from peptide mixtures for peptide therapeutics using combinations of natural and non-natural amino acid alkyl ester monomers and specific combinations of these monomers as dimers, trimers and higher oligomers, the peptide mixtures having useful physical and/or biological properties, the method of peptide synthesis comprising the steps of:
a) admixing at least one natural and at least one non-natural amino acid alkyl ester monomer, dimer, trimer, or higher oligomers with at least one enzyme in a reaction medium;
b) initiating a reaction by heating the mixture to between about 5° C. to about 90° C. for between 5 minutes and 24 hours; and
c) recovering the oligopeptide.

2. The process as claimed in claim 1 wherein the amino acid alkyl ester has the structure:
H₂N—[CH(R)]_{d}—(CR′H)ₑ—COOX (1)
wherein
R represents an amino acid side chain,
R′ represents a different amino acid side chain different from R, and
X is a straight or branched chain alkyl ester consisting of an alkyl group selected from those containing from 1 to 20 carbon atoms, or is an activated ester.

3. The process as claimed in claim 2, wherein the alkyl ester is selected from the group consisting of methyl, ethyl, and propyl groups.

4. The process as claimed in claim 2, wherein X is a straight or branched chain alkyl ester consisting of an alkyl group selected from those containing from 1 to 6 carbon atoms.

5. The process as claimed in claim 2, wherein the activated ester is selected from the group consisting of guanadinophenyl, p-nitrophenyl, 1,1,1,3,3,3,-hexafluoroisopropyl, 2,2,2-triifluoroethyl, 2-chloro ethyl ester, carbamoyl methyl ester, benzyl esters, and anilides.

6. The process as claimed in claim 2, wherein structure 1 has e=0, d=1, the stereochemistry is L, and R is selected from the group of natural amino acid side chains consisting of glycine, alanine, valine, leucine, isoleucine, serine, threonine, cysteine, methionine, proline, phenylalanine, tyrosine, tryptophan, histidine, lysine, arginine, aspartic acid, asparagine, glutamic acid, glutamine, and combinations thereof.

7. The process as claimed in claim 2, wherein structure 1 is selected from the family of non-natural amino acids and β-amino acids having the structure:
H₂NCH₂CH(R)—COOX (2)
wherein the β-amino acids are useful substrates for protease-catalyzed oligopeptide synthesis or protease-catalyzed coupling of preformed segments of oligo(amino acids).

8. The process as claimed in claim 7, wherein the β-amino acids are selected from the group consisting of β-alanine, L-β-homotyrosine, L-β-homoleucine, L-β-homoisoleucine and L-β-homotryptophan, and wherein the non-natural amino acid esters are selected from the group consisting of carnitine [3-hydroxy-4-trimethylammonio-butanoate], ornithine [(+)-(S)-2,5-diamino valeric acid], citruline [2-amino-5-(carbamoylamino)pentanoic acid], and 4-aminobutanoic acid and L-dopamine.

9. The process as claimed in claim 2 wherein structure 1 has e=0, R=H, and wherein d=1 to 10.

10. The process as claimed in claim 9, wherein d=1 to 5.

11. The process as claimed in claim 2, wherein X is ethyl.

12. The process as claimed in claim 1, wherein the reaction medium comprises a phosphate, acetate, borate, carbonate, HEPES, an amine to maintain reaction medium pH, or sulphate buffers with a concentration of between 0.1 M to 1.5 M.

13. The process as claimed in claim 12, further comprising a water-miscible cosolvent selected from the group consisting of formamides, alcohols (1°, 2°, 3°), dimethyl sulfoxide, tetrahydrofuran, acetone, acetonitrile, 1,2-ethylene glycol, 1,3-propylene glycol, or 1,4-butanediol is added in concentrations from 0 to 50%-v/v.

14. The process as claimed in claim 1, wherein the enzyme is selected from a member of a hydrolytic enzyme family comprising proteases, lipases, esterases and cutinases.

15. The process as claimed in claim 14, wherein the enzyme is a protease selected from the group consisting of papain, bromelain, α-chymotrypsin, trypsin, Multifect P-3000 (Genencor), Purafect prime L (Genencor), alkaline protease (Genencor), metalloprotease (thermolysin), protease from subtilisin (family), pronase 1, glutaminase, carboxypeptidase Y, clostrapin, protease from Aspergillus oryzae species, pepsin, cathepsin, ficin, alcalase, carboxypeptidase, calpains, actinidin, chymosin, carbonic anhydrase, nonribosomal peptide synthetase, thrombin, cardosins A or B, pronase, and combinations thereof,

16. The process as claimed in claim 1, wherein the enzyme is added to the reaction medium as enzyme powders, in solution, or immobilized on a support.

17. The process as claimed in claim 1, wherein the reaction is terminated by separation of the oligopeptide as a formed precipitated functionalized oligopeptide product by filtration or centrifugation from the enzyme remaining in the reaction medium.

18. The process as claimed in claim 1, wherein the reaction is terminated by using a membrane with a suitable pore size that separates the oligopeptide as a soluble oligopeptide product from the soluble enzyme.

19. The process as claimed in claim 1, wherein the reaction is terminated by selective precipitation of either a soluble enzyme or a soluble oligopeptide product.

20. The process as claimed in claim 1, wherein the reaction time is between 5 minutes and 24 hours.

21. The process as claimed in claim 1, wherein the reaction time is between 10 minutes and 8 hours.

22. The process as claimed in claim 1, wherein the reaction time is between 30 minutes and 3 hours.

23. The process as claimed in claim 1, wherein the reaction temperature is between 5° C. and 90° C.

24. The process as claimed in claim 1, wherein the reaction temperature is between 25° C. and 60° C.

25. The process as claimed in claim 1, wherein the reaction temperature is between 30° C. and 40° C.

26. The process as claimed in claim 1, wherein the reaction is performed by passing reactants through a column wherein the stationary phase consists of the immobilized enzyme.

27. The process as claimed in claim 1, wherein the oligopeptide consists of a mixture of oligomers where the average chain length, determined by measuring the number average molecular weight, ranges from 2 to 100 units.

28. The process as claimed in claim 1, wherein the oligopeptide consists of a mixture of oligomers where the average chain length, determined by measuring the number average molecular weight, ranges from 5 to 50 units.

29. The process as claimed in claim 1, wherein the oligopeptide consists of a mixture of oligomers where the average chain length, determined by measuring the number average molecular weight, ranges from 10 to 20 units.

30. The process as claimed in claim 1, wherein the oligopeptide consists of a mixture of oligomers with a polydispersity, determined by dividing the weight average molecular weight by the number average molecular weight, of 50.

31. The process as claimed in claim 1, wherein the oligopeptide consists of a mixture of oligomers with a polydispersity, determined by dividing the weight average molecular weight by the number average molecular weight, of less than 25.

32. The process as claimed in claim 1, wherein the oligopeptide consists of a mixture of oligomers with a polydispersity, determined by dividing the weight average molecular weight by the number average molecular weight, of less than 5.

33. The process as claimed in claim 1, wherein the oligopeptide consists of a mixture of oligomers with a polydispersity, determined by dividing the weight average molecular weight by the number average molecular weight, or less than 1.5.

34. The process as claimed in claim 1, wherein the oligomers are useful for metal binding and are prepared from co-oligomerization of amino acid 1 (AA1) and amino acid 2 (AA2), wherein AA1 is L-Et₂-glutamic acid and AA2 is the ethyl ester derivative selected from the group consisting of L-histidine, L-cysteine, L-lysine, L-asparagine, or L-aspartic acid.

35. The process as claimed in claim 34, wherein AA2 is L-cysteine ethyl ester.

36. The process as claimed in claim 34, wherein the oligopeptide comprises from 30 to 70 mol % of L-γ-Et-glutamic acid units and 70 to 30 mol % of L-cysteine units.

37. The process as claimed in claim 34, wherein the oligopeptide comprises from 40 to 60 mol % of L-γ-Et-glutamic acid units and 60 to 40 mol % of L-cysteine units.

38. The process as claimed in claim 34, wherein the oligopeptide comprises from 45 to 55 mol % of L-γ-Et-glutamic acid units and 55 to 45 mol % of L-cysteine units.

39. The process as claimed in claim 1, wherein the oligomers are useful for antimicrobial activity and are prepared from co-oligomerization of amino acid 1 (AA1) and amino acid 2 (AA2), wherein AA1 is L-lysine ethyl ester or L-arginine ethyl ester and AA2 is the ethyl ester derivative selected from the group consisting of L-alanine, L-valine, L-leucine, L-isoleucine, or L-phenylalanine.

40. The process as claimed in claim 39, wherein the oligopeptide comprises from 20 to 50 mol % of AA1 units and 80 to 50 mol % of AA2 units.

41. The process as claimed in claim 39, wherein the oligopeptide comprises from 30 to 50 mol % of AA1 units and 70 to 30 mol % of AA2 units.

42. The process as claimed in claim 39, wherein the oligopeptide comprises from 40 to 50 mol % of AA1 units and 60 to 50 mol % of AA2 units.

43. The process as claimed in claim 1, further comprising fractionation of synthesized co-oligomer mixtures to obtain a product mixture with enhanced physical or biological activity.

44. The process as claimed in claim 43, wherein the fractionation is achieved by centrifugation filters with predefined molecular cut-off values to obtain desired product fractions.

45. The process as claimed in claim 43, wherein the fractionation is achieved by differential solubility using common organic solvents selected from the group consisting of methanol, ethanol, 1-propanol, isopropanol, acentonitrile, 1,4-dioxane, chloroform, THF, DMSO and DMF, and combinations thereof.

46. The process as claimed in claim 43, wherein the fractionation is achieved by shifts in solution pH with or without variation in the ionicity or nature of the cationic species.

47. The process as claimed in claim 43, wherein the fractionation is achieved by exploiting different molecular weights or hydrodynamic volumes of constituents in the product mixture and such fractionation is achieved by size exclusion chromatograph (SEC).

48. The process as claimed in claim 1, wherein the natural and non-natural amino acid alkyl esters are selected from the group of structural motifs of metal binding, adhesion, self-assembly, antimicrobial activity, protein inhibition, ingredients in cosmetic formulations, and peptide therapeutics.

49. The process as claimed in claim 1, further comprising adding a molecule to the mixture of reactants that end-functionalizes the N-terminus, C-terminus or both ends of the peptide, wherein the general formula for the synthesized oligomer is C-peptide-B wherein B is a group at the carboxyl terminus and C is a group at the N-terminus.

50. The process as claimed in claim 49 wherein the end-functionalization agent comprises an activated ester with the structure:
Y′Y—[H]ₐN—CH(R)—(CR′H)ₙ—COOX (3)
wherein
X is a straight or branched chain alkyl ester consisting of an alkyl group selected from those containing from 1 to 20 carbon atoms, or is an activated ester,
Y and/or Y′ is selected from H, methyl, ethyl, CH₂═CH—CO—, CH₂═C(CH₃)—CO—, HOOC—CH═CH—CO— (cis or trans), functional groups used for photolytic crosslinking, cinnamoyl (Ph-CH═CH—CO—) group, groups that are crosslinkable via redox catalysts, and HO-Ph-(CH₂)—CO— where the hydroxyl group is at the para-position.

51. The process as claimed in claim 50, wherein Y and/or Y′ is selected from structures that are used in bioconjugate chemistry.

52. The process as claimed in claim 51, wherein Y and/or Y′ is selected from the group consisting of alkyne functionalized molecules, azide functionalized molecules, and terminal alkene functionalized molecules.

53. The process as claimed in claim 51, wherein Y and/or Y′ is selected from the group consisting of 4-alkyne-pentanoate (HC≡C—CH₂—CH₂—CO—), N₃—CH₂—CH₂—CH₂—CH₂—CH₂—CO—), and 2-propen-1-amine.

54. The process as claimed in claim 51, wherein n=0, Y is H, Y′ is CH₂═CH—CO— or CH₂═C(CH₃)—CO—, and R is selected from the group consisting of glycine, alanine, valine, leucine, isoleucine, serine, threonine, cysteine, methionine, proline, phenylalanine, tyrosine, tryptophan, histidine, lysine, arginine, aspartic acid, asparagine, glutamic acid, glutamine, and combinations thereof.

55. A process for preparing oligopeptides end-functionalized at the N-terminus, C-terminus or at both ends and that has the general formula C-peptide-B, wherein B is a group at the carboxyl terminus, C is a group at the N-terminus, comprising the steps of:
a) admixing at least one natural and at least one non-natural amino acid alkyl ester monomer, dimer, trimer, or higher oligomers with at least one enzyme in a reaction medium;
b) initiating a reaction by heating the mixture to between about 5° C. to about 90° C. for between 5 minutes and 24 hours; and
c) recovering the oligopeptide.

56. The process as claimed in claim 55, further comprising performing a modification of the N-terminal amino group by conventional coupling methods using conventional chemical methods.

57. The process as claimed in claim 56, wherein the N-terminal group of oligopeptides is modified by N-acylation chemistry using conventional chemical methods.

58. The process as claimed in claim 57, wherein the N-acylated amino acids formed has the structure:
R(C═O)NH-peptide-COOX (4)
wherein R(C═O) is derived from a natural fatty acid selected from the group consisting of lauric acid (dodecanoic acid), myristic acid (tetradecanoic acid), palmitic acid (hexadecanoic acid), palmitoleic acid (9-hexadecenoic acid), stearic acid (octadecanoic acid), oleic acid (9-octadecenoic acid), ricinoleic acid (12-hydroxy-9-octadecenoic acid), linoleic acid (9,12-octadecadienoic acid), α-linolenic acid (9,12,15-octadecatrienoic acid), γ-linolenic acid (6,9,12-octadecatrienoic acid), behenic acid (docosanoic acid), and erucic acid (13-docosenoic acid).

59. The process as claimed in claim 58, wherein the fatty acid is first modified by hydrogenation, epoxidation, or hydroxylation prior to reaction with NH₂ terminal groups of oligopeptides.

60. The process as claimed in claim 58, wherein R is selected from the group consisting of —CH₂═CH—CO—, CH₂═C(CH₃)—CO—, and HOOC—CH═CH—CO— (cis or trans).

61. The process as claimed in claim 55, wherein the end-functionalization agent comprises an amine having the structure:
H₂N—CH(R)—(CR′H)ₙ—COHN—Z (3)
wherein n=0,
wherein R is selected from the group consisting of lauric acid (dodecanoic acid), myristic acid (tetradecanoic acid), palmitic acid (hexadecanoic acid), palmitoleic acid (9-hexadecenoic acid), stearic acid (octadecanoic acid), oleic acid (9-octadecenoic acid), ricinoleic acid (12-hydroxy-9-octadecenoic acid), linoleic acid (9,12-octadecadienoic acid), α-linolenic acid (9,12,15-octadecatrienoic acid), γ-linolenic acid (6,9,12-octadecatrienoic acid), behenic acid (docosanoic acid), and erucic acid (13-docosenoic acid), and
wherein Z is selected from structures that are used in bioconjugate chemistry.

62. The process as claimed in claim 61, wherein Z is selected from the group consisting of maleimide functionalized molecules for thiol-maleimide chemistry, azide functionalized molecules for azide alkyne chemistry, alkyne functionalized molecules for azide alkyne chemistry.

63. The process as claimed in claim 62, wherein Z is selected from the group consisting of —CH₂—CH₂-maleimide, —CH₂—CH₂—N₃), and —CH₂≡CH.

64. The process as claimed in claim 1, wherein a specific combination of two amino acids is selected from the natural and non-natural amino acids based on synthesizing oligopeptide mixtures by protease catalysis for a desired structural motif, the dimer of the two amino acids selected is synthesized by chemical or enzymatic methods, and then the dimer alkyl ester is used as monomer to prepare alternating co-oligopeptides.

65. The process as claimed in claim 1, wherein dimers of L-Ala and L-Gly are prepared and then the corresponding dipeptide ethyl ester is oligomerized by protease catalysis to form alternating peptides of the composition -(-L-Ala-L-Gly-)ₙ-.

66. The process as claimed in claim 65 where L-Ala-L-Gly ethyl ester is prepared by esterification of L-Ala-L-Gly in ethanol with thionyl chloride.

67. The process as claimed in claim 1, wherein dimers of L-Lys and L-Leu are prepared and then the corresponding dipeptide ethyl ester is oligomerized by protease catalysis to form alternating peptides of the composition -(-L-Lys-L-Leu-)ₙ-

68. The process as claimed in claim 67, wherein Boc-L-Lys(Boc)-L-Leu-ethyl ester is prepared by coupling Boc-Lys(Boc)-OSu with leucine ethyl ester at room temperature catalyzed by triethylamine.

69. The process as claimed in claim 68, wherein Boc-L-Lys(Boc)-L-Leu-ethyl ester is deprotected by using dichloromethane (DCM)/trifluoroacetic acid(TFA) as co-solvents in a 3:1 v/v ratio.

70. The process as claimed in claim 1, wherein dimers of L-Lys and L-Phe are prepared and then the corresponding dipeptide ethyl ester is oligomerized by protease catalysis to form alternating peptides of composition -(-L-Lys-L-Phe-)ₙ-.

71. The process as claimed in claim 70, wherein Boc-L-Lys(Boc)-L-Phe-ethyl ester is prepared by coupling Boc-Lys(Boc)-OSu with phenylalanine ethyl ester at room temperature catalyzed by triethylamine.

72. The process as claimed in claim 71, wherein Boc-L-Lys(Boc)-L-Phe-ethyl ester is deprotected by using dichloromethane (DCM)/trifluoroacetic acid(TFA) as co-solvents in a 3:1 v/v ratio.

73. The process as claimed in claim 55, wherein the end-functionalization agent comprises an amine having the structure:
H₂N—CH(R)—(CR′H)ₙ—COHN—Z (3)
and an activated ester having the structure:
Y′Y—[H]ₐN—CH(R)—(CR′H)ₙ—COOX
wherein n=0,
wherein R is selected from the group consisting of glycine, alanine, valine, leucine, isoleucine, serine, threonine, cysteine, methionine, proline, phenylalanine, tyrosine, tryptophan, histidine, lysine, arginine, aspartic acid, asparagine, glutamic acid, glutamine, and combinations thereof, and
wherein Z is selected from the group consisting of an azide functionalized moiety,
wherein Y is H, and
wherein Y′ is an azide functionalized moiety or an alkyne functionalized moiety.

74. The process as claimed in claim 73, wherein Z and Y are selected to be used in combination to prepare oligopeptides that are functionalized at both the amino- and carboxyl-termini.

75. The process as claimed in claim 73, wherein Z is —CH₂—CH₂—N₃), Y is H, and Y′ is N₃—CH₂—CH₂—CH₂—, CH₂—CH₂≡CH, or HC≡C—CH₂—CH₂—CO—.

76. The process as claimed in claim 55, wherein an oligopeptide mixture, synthesized by protease-catalysis, is functionalized at either the N- or C-terminus with groups useful in bioconjugate chemistry, and end-functionalized groups are used to conjugate the peptides to substances.

77. The process as claimed in claim 55, further comprising:
functionalizing an oligopeptide mixture, synthesized by protease-catalysis, at either the N- or C-terminus with groups useful in bioconjugate chemistry, and
coupling end-functionalized peptides to chain segments of natural, synthetic or hybrid polymer chains,
thereby resulting in coupled products consisting of:
peptide mixtures;
peptides of uniform chain length and sequence synthesized by chemical methods, isolated from nature, or produced by recombinant DNA methods;
synthetic polymers such as heterobifunctional polyethylene glycol; and
chain segments of DNA; and oligo- or polysaccharides such as those belonging to members of the glycosaminoglycan family, chitosan, pectin and amylose.

78. The process as claimed in claim 55, further comprising:
functionalizing an oligopeptide mixture, synthesized by protease-catalysis, at both the N- and C-terminus with groups useful for bioconjugate chemistry, and
copolymerizing end-functionalized peptides with at least one chain segments that has suitable functional groups at both chain termini selected from the group consisting of:
peptides of uniform chain length and sequence synthesized by chemical methods, isolated from nature, or produced by recombinant DNA methods;
synthetic polymers such as end-functionalized polyethylene glycol, oligo- or polylactic acid, oligo- or polythiopene;
chain segments of DNA; and
oligo- or polysaccharides such as those belonging to members of the glycosaminoglycan family, chitosan, pectin and amylose.
